(19) United States
(12) Patent Application Publication
(10) Pub. No.: US 2016/0250045 A1
(43) Pub. Date: Sep. 1, 2016

(54) PROSTHETIC DEVICE UTILIZING ELECTRIC VACUUM PUMP

(71) Applicant: The Ohio Willow Wood Company, Mount Sterling, OH (US)

(72) Inventors: James M. Colvin, Hilliard, OH (US); Michael L. Haynes, Columbus, OH (US); Mark W. Ford, Jamestown, OH (US); Mark W. Groves, Colubus, OH (US); Jeffrey A. Denune, Galloway, OH (US)

(21) Appl. No.: 15/149,636

(22) Filed: May 9, 2016

Related U.S. Application Data
(63) Continuation-in-part of application No. 13/231,690, filed on Sep. 13, 2011, now Pat. No. 9,333,098.
Application No. 13/231,690 is a division of application No. 11/688,402, filed on Mar. 20, 2007, now Pat. No. 8,016,892.
Application No. 11/688,402 is a continuation of application No. 11/423,632, filed on Jun. 12, 2006, now Pat. No. 7,947,085.
Application No. 11/423,632 is a continuation-in-part of application No. 11/149,858, filed on Jun. 10, 2005, now Pat. No. 7,914,586.

(51) Int. Cl. A61F 2/80

(52) U.S. Cl. CPC A61F 2/80; A61F 2002/805; A61F 2002/802; A61F 2250/0002

(57) ABSTRACT

According to the embodiments described herein, prosthetic devices can include a prosthetic socket, a vacuum passage, and an evacuation device. The interior of the prosthetic socket can be adapted to receive a residual limb. The vacuum passage can extend through the side wall of the prosthetic socket and into the interior of the prosthetic socket. The evacuation device can include an electrically powered vacuum pump contained within a housing. The housing can be attached to the side wall on the exterior of the prosthetic socket. The electrically powered vacuum pump can be in communication with the vacuum passage. The electrically powered vacuum pump can draw air from the interior of the prosthetic socket, while the residual limb is received within the interior of the prosthetic socket, to evacuate the prosthetic socket.

### CROSS-REFERENCE TO RELATED APPLICATIONS

The present application is a continuation-in-part of U.S. patent application Ser. No. 13/231,690, filed on Sep. 13, 2011, now U.S. Pat. No. 9,333,098, issued May 10, 2016, which is a division of U.S. patent application Ser. No. 11/688,402, filed on Mar. 20, 2007, now U.S. Pat. No. 8,016,892, issued Sep. 13, 2011, which is a continuation-in-part of U.S. patent application Ser. No. 11/423,632, filed on Jun. 12, 2006, now U.S. Pat. No. 7,947,085, issued May 24, 2011, which is a continuation-in-part of U.S. patent application Ser. No. 11/149,858, filed on Jun. 10, 2005, now U.S. Pat. No. 7,914,586 issued May 29, 2011.

### BACKGROUND

The present disclosure is directed to electrically-powered evacuation devices for use in evacuating a prosthetic socket and/or to prosthetic limbs incorporating such electrically-powered evacuation devices. The present disclosure is also directed to various systems and methods for configuring, monitoring, performing, adjusting and controlling such devices.

Artificial limbs have been in use throughout history, having been first recorded circa 2750 B.C. During that period of time, interfacing and suspending an artificial limb has been a continuing challenge. Various and numerous theories and anatomical constructs have been used over time in an evolving manner, and these have revealed a number of key factors in maximizing comfort and functional potential for persons who wear artificial limbs.

Firstly, the surgical procedure used to perform limb amputation is an important factor. The size and shaping of the patient's residual limb can affect the comfort the patient will later have with a prosthesis. Stated simply, the residual limb and prosthesis can be configured to interface tightly and couple and distribute pressure evenly across the surface of the residual limb.

Early versions of artificial limbs required the use of leather or equivalent straps or belts to suspend the artificial limb upon the person. Later systems employed linkage techniques such as condylar wedges, rubber or synthetic elastic tubing, thermoplastic roll-on sleeves with pin locking systems, and sub-atmospheric pressure. Of these, sub atmospheric pressure can create a linkage that improves proprioceptive feedback and control for the artificial limb user. Sub atmospheric pressure can also improve the linkage between the user's limb and the prosthetic device.

Creating a reliable sub atmospheric pressure chamber between the residual limb and prosthetic device has, however, proved to be a challenge. As new airtight thermoplastic and thermoset materials have evolved, along with airtight thermoplastic roll-on liners, the potential for creating a sub-atmospheric pressure within the prosthetic chamber (socket) has improved. Specifically, the patient's residual limb can be covered with a roll-on urethane, silicone, or other thermoplastic or thermoset liner, which can protect the user's tissue from unwanted isolated high negative pressure values, and provides cushioning for the tissue at the same time. The liner can also distribute the sub-atmospheric pressure applied to the user's limb in a more uniform manner.

Several mechanical means for creating an elevated negative pressure chamber within a prosthetic socket have emerged. One method disclosed in U.S. Pat. No. 6,554,868, utilizes a weight activated pump, in which sub atmospheric pressure can be maintained strategically within the socket as the user walks. Under this approach, vacuum can be maintained as the patient ambulates with the artificial limb.

This method of evacuating a prosthetic socket has several disadvantages, however. First, the weight activated pump can be heavy, and can be difficult to remove even in the case of a pump failure. The weight activated pump also can require a certain minimum space between the user's limb and prosthetic foot, which may be more than is available if the patient has a relatively long residual limb. This can prohibit the use of this technology for many artificial limb users. Further, a weight-activated pump system can require some number of weight activated strokes before becoming effective.

Another evacuation method disclosed in the above-referenced patent uses a hand-held sub-atmospheric pressure pump, much like that used to bleed brake systems on an automobile. This method can provide socket evacuation, but can require the individual to carry the hand-held pump upon their person for use in case of vacuum failure. The hand-held pump can be awkward for many individuals to use and can require a certain amount of dexterity and strength to operate. This can be difficult for elderly individuals.

As can be understood from the foregoing discussion, mechanical systems for evacuating a prosthetic socket can have several disadvantages. Aside from those specific disadvantages detailed above, such mechanical systems can be further burdened with other general problems. For example, the evacuation pump associated with such systems can only be activated when the user is ambulating, and then can remain activated with every step—regardless of the wishes of the user.

Therefore, one general disadvantage to such mechanical systems is that the pump can be unable to draw vacuum for a sedentary user. Accordingly, absent carrying and using a separate hand-held pump, properly donning an associated prosthesis can require standing up and walking on the prosthesis in a partially donned (i.e., non-evacuated) state. Similarly, if the socket loses pressure while the user is sitting or otherwise non-ambulatory, a separate hand-held pump can be required to re-evacuate the socket, or the user can be required to walk or bounce on an improperly suspended prosthesis in order to re-evacuate the socket.

Another disadvantage to such mechanical evacuation systems is that a weight-activated pump can be configured to evacuate the prosthetic socket to some predetermined level. As such, a user may not be permitted to adjust the level of vacuum to coincide with a particular activity or comfort level. For example, the predetermined level may not be configured for a period of increased activity, nor to compensate for a particularly sore or sensitive residual limb.

Furthermore, known evacuation systems can be bulky, unattractive, and difficult to cosmetically finish. For example, the bulkiness can make it difficult to apply a cosmetic cover that imparts a lifelike appearance. Also, applying a cosmetic cover may interfere with the function of the evacuation system or may prevent or discourage recommended access to the evacuation system.

Thus, there is a need for additional means of achieving sub-atmospheric pressure within a prosthetic socket.

### SUMMARY

The present disclosure overcomes the disadvantages inherent to prosthetic socket evacuation devices using mechanical (e.g., weight-activated) pumps. Rather, the present disclosure is directed to socket evacuation devices employing an electrically-activated pump. Because the electrically-activated pump does not require manual manipulation to create vacuum, ease of use can be improved compared to a manual pump. Further, the evacuation devices described herein can be relatively compact in size and can have elements the reduce power consumption. Accordingly, the evacuation devices may be readily incorporated into/onto a prosthesis.

The embodiments describe herein afford advantages over manual pumps and gait-driven pumps. For example, the embodiments described herein relate to practical approaches to providing an electrically evacuated prosthetic device. The '868 patent referenced above suggests the inclusion of a generically drawn “vacuum source” and “power source”, and a regulator for automatic vacuum maintenance, into an outer socket of a prosthesis (see, e.g., FIGS. 7 and 9 and discuss thereof); however, the '868 patent fails to suggest a vacuum source or power source that is of suitable size and weight. The present disclosure thus represents an advance and enabled approaches to providing an electrically actuated, portable vacuum pump in a prosthesis.

An electrically-activated evacuation device described herein offers advantages compared to manual or gait-driven devices. For example, in addition to embodiments wherein the vacuum level is directly controlled by the user, the embodiments described herein further relate to automatic or automatic vacuum level control and/or semi-automatic or automatic vacuum regulation.

Additionally, electrically-activated evacuation devices of the present disclosure can be made to blend in with the rest of a prosthesis, and can be integrated into the prosthesis—improving the ability to cosmetically finish the prosthesis, if so desired. Even in embodiments using wireless capabilities, applying a cosmetic cover can be provided without interfering with the function of the evacuation system.

In one embodiment, a prosthetic device can include a prosthetic socket, a vacuum passage, and an evacuation device. The prosthetic socket can include a side wall extending from a distal end of the prosthetic socket to form an interior and an exterior of the prosthetic socket. The interior of the prosthetic socket can be adapted to receive a residual limb. The vacuum passage can extend through the side wall of the prosthetic socket and into the interior of the prosthetic socket. The evacuation device can include an electrically powered vacuum pump and a source of electric power both contained within a common housing. The common housing can be attached to the side wall on the exterior of the prosthetic socket. The electrically powered vacuum pump can be in communication with the vacuum passage. The electrically powered vacuum pump can draw air from the interior of the prosthetic socket, while the residual limb is received within the interior of the prosthetic socket, to evacuate the prosthetic socket.

In another embodiment, a prosthetic device can include a prosthetic socket, a mounting adapter, a vacuum passage, and an evacuation device. The prosthetic socket can include a side wall extending from a distal end of the prosthetic socket to form an interior and an exterior of the prosthetic socket. The interior of the prosthetic socket can be adapted to receive a residual limb. The mounting adapter can be built directly into the side wall of the prosthetic socket. The vacuum passage can extend into the interior of the prosthetic socket and through the side wall of the prosthetic socket and the mounting adapter. The evacuation device can include an electrically powered vacuum pump and a source of electric power both contained within a common housing. The common housing can be on the exterior of the prosthetic socket and attached to the mounting adapter. The electrically powered vacuum pump can be in communication with the vacuum passage. The electrically powered vacuum pump can draw air from the interior of the prosthetic socket, while the residual limb is received within the interior of the prosthetic socket, to evacuate the prosthetic socket.

In another embodiment, a prosthetic device can include a prosthetic socket, a mounting adapter, a vacuum passage, and an evacuation device. The prosthetic socket can include a side wall extending from a distal end of the prosthetic socket to form an interior and an exterior of the prosthetic socket. The interior of the prosthetic socket can be adapted to receive a residual limb. The mounting adapter can be built directly into the side wall of the prosthetic socket. The vacuum passage can extend into the interior of the prosthetic socket and through the side wall of the prosthetic socket and the mounting adapter. The evacuation device can include an electrically powered vacuum pump, a source of electric power, and an evacuation device vacuum passage each contained within a common housing. The common housing can be on the exterior of the prosthetic socket and attached to the mounting adapter. The evacuation device vacuum passage can be aligned with the vacuum passage that extends through the side wall of the prosthetic socket and the mounting adapter. The electrically powered vacuum pump can be in communication with the vacuum passage. The electrically powered vacuum pump can draw air from the interior of the prosthetic socket, while the residual limb is received within the interior of the prosthetic socket, to evacuate the prosthetic socket.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a prosthetic limb incorporating an electric vacuum pump according to one or more embodiments shown and described herein;

FIG. 2 schematically depicts a disassembled view of the prosthetic limb of FIG. 1, illustrating internal components thereof, according to one or more embodiments shown and described herein;

FIG. 3 schematically depicts a cutaway view of the prosthetic limb of FIG. 1 showing the internal components as positioned when the limb is in use according to one or more embodiments shown and described herein;

FIGS. 4A and 4B schematically depict cutaway views of the prosthetic limb of FIG. 1 showing its use in creating vacuum engagement of a limb with a socket according to one or more embodiments shown and described herein;

FIG. 5 schematically depicts an electric pump and power source housed in a separate portable evacuation device according to one or more embodiments shown and described herein;

FIG. 6 schematically depicts an electric pump and power source placed into a sleeve that is subsequently installed into a pylon according to one or more embodiments shown and described herein;

FIG. 7 schematically depicts a prosthetic limb employing an evacuation device that includes a vacuum pump and power source located within a housing designed for attachment to a universal distal adapter that is built into the distal end of a prosthetic socket according to one or more embodiments shown and described herein;

FIG. 8 schematically depicts a plan view into the socket of the prosthetic limb of FIG. 7, wherein a portion of the universal distal adapter and a portion of the housing are visible according to one or more embodiments shown and described herein;

FIG. 9 schematically depicts a section view of a portion of the prosthetic limb of FIG. 7, taken along line C-C of FIG. 8 according to one or more embodiments shown and described herein;

FIG. 10A schematically depicts an enlarged view of the detailed area called out in FIG. 9 according to one or more embodiments shown and described herein;

FIG. 10B schematically depicts a bottom plan view of the universal distal adapter according to one or more embodiments shown and described herein;

FIG. 11 schematically depicts a section view of a portion of the prosthetic limb of FIG. 7, taken along line D-D of FIG. 8 according to one or more embodiments shown and described herein;

FIG. 12 schematically depicts an enlarged view of the detailed area called out in FIG. 11 according to one or more embodiments shown and described herein;

FIG. 13 schematically depicts an evacuation device including a vacuum pump and power source located within a housing that is mounted around the pylon of a prosthetic limb according to one or more embodiments shown and described herein;

FIG. 14A schematically depicts an evacuation device including a vacuum pump and power source located in a housing that is attached to an adapter integrated into a side wall of a prosthetic socket according to one or more embodiments shown and described herein;

FIG. 14B schematically depicts an evacuation device including a vacuum pump and power source located in a chamber that is integral to and protrudes from the side wall of a prosthetic socket according to one or more embodiments shown and described herein;

FIG. 15 schematically depicts an evacuation device including a vacuum pump and power source located in a housing that is positioned within an exoskeletal prosthetic device according to one or more embodiments shown and described herein;

FIG. 16 schematically depicts an evacuation device including a vacuum pump and power source located in a housing that is affixed to a mounting plate designed to be mounted between adjacent components of a prosthetic limb according to one or more embodiments shown and described herein;

FIG. 17 schematically depicts an evacuation device including a vacuum pump and power source located in a prosthetic foot or within a housing that is positioned in a prosthetic foot according to one or more embodiments shown and described herein;

FIG. 18 schematically depicts an evacuation device including a vacuum pump and power source located within a housing that is located on the user's person and provided to evacuate the socket of a prosthetic limb according to one or more embodiments shown and described herein;

FIG. 19 schematically depicts a manifold that connects a vacuum source to the interior of a prosthetic socket according to one or more embodiments shown and described herein;

FIG. 20 schematically depicts a magnetic switch that can be used to initiate the energizing of a vacuum pump according to one or more embodiments shown and described herein;

FIG. 21 schematically depicts a cross-sectional view showing a portion of a prosthetic limb including a vacuum pump and power source that are located within a housing designed for attachment to a universal distal adapter that is built into the distal end of a prosthetic socket according to one or more embodiments shown and described herein; and

FIGS. 22a-22b schematically depict an electronic vacuum control system that includes a handheld controller wirelessly connected to a vacuum control assembly according to one or more embodiments shown and described herein.

### DETAILED DESCRIPTION

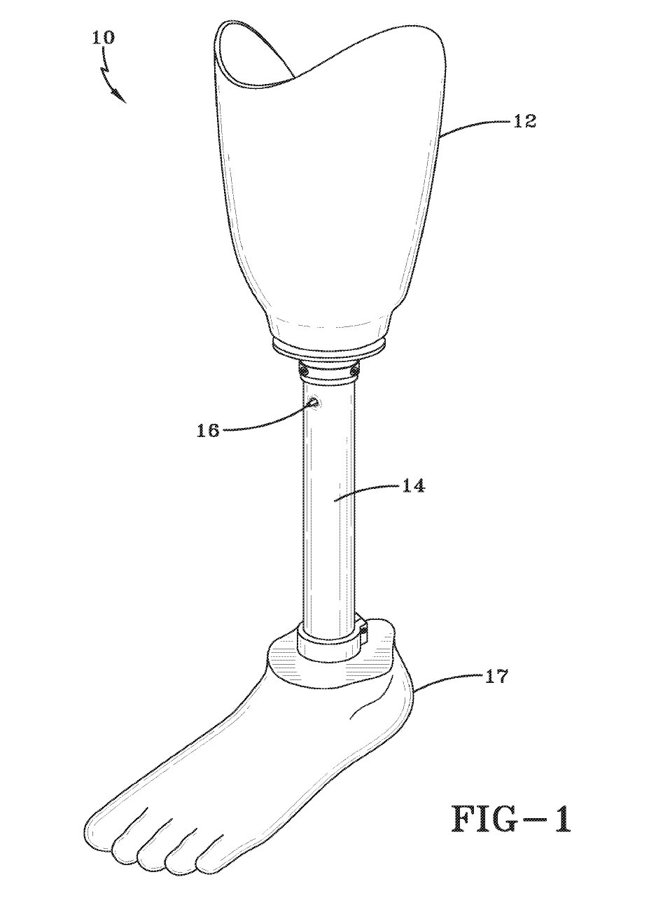

Referring now to, FIG. 1 a prosthesis 10 can include a socket 12 for receiving an amputee's residual limb, a column (pylon) 14, which can be a cylindrical section of lightweight metal such as aluminum, and an artificial foot 17. As can be seen in FIG. 1, the pylon 14 can include a vacuum actuator button 16 used to actuate an electric vacuum pump within the pylon that draws air from the socket 12 and, as a result, draws the residual limb into intimate contact with the interior of the socket 12.

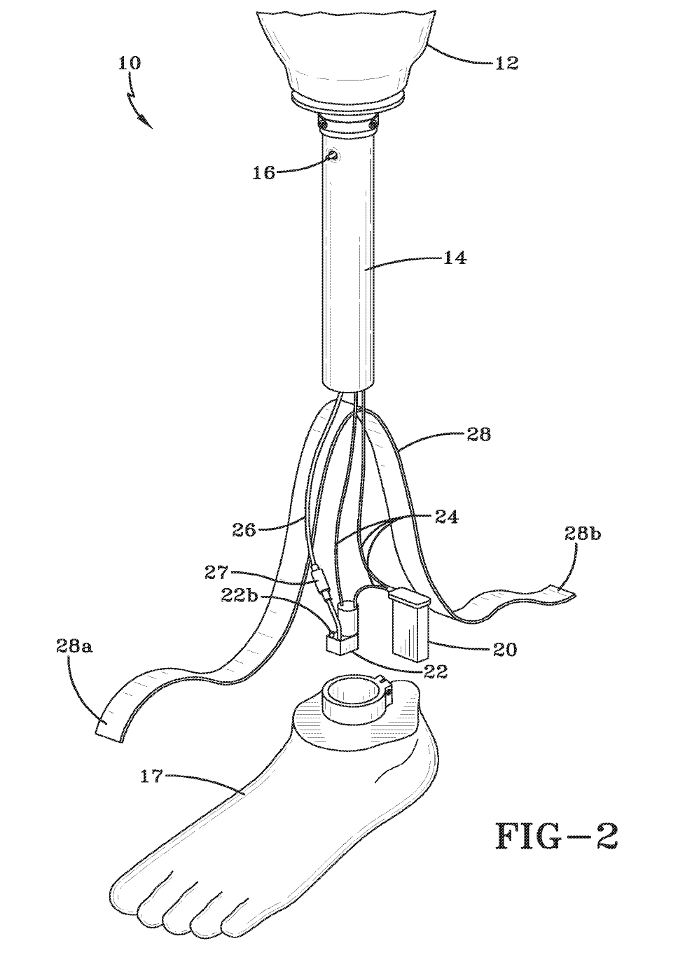

FIG. 2 schematically depicts the prosthesis 10 of FIG. 1 in a disassembled state to show the component parts within the pylon 14. Internal to the pylon 14 can be a power source 20, such as a capacitor or a conventional 9-volt battery, a vacuum pump 22, and electrical lines 24 for delivering electrical power from power source 20 to vacuum pump 22, and vacuum line 26 for drawing vacuum from socket 12 through a check valve 27. The power source 20, vacuum pump 22, electrical lines 24, vacuum line 26 and check valve 27 components can be inserted into the pylon 14 after insertion of a ribbon 28. The ribbon 28 may be subsequently used to extract the components (e.g., for changing or recharging the power source 20).

One suitable type of vacuum pump for use in the present embodiments can include the model VMP 1624 Series of vacuum pumps, available from Virtual Industries, Inc., 2130 Vector Place, Colorado Springs Colo. A specific model that can be particularly suitable for application, as shown herein, is model 1624-009-S. Suitable pumps can be capable of drawing vacuum up to 18 inches of mercury (−594 millibar), which is sufficient for use in a prosthesis. The pump flow rate can be as large as 1300 ml per minute. The voltage for the specific model identified above is 9 volts, permitting use of the pump with a conventional disposable or rechargeable 9-volt battery. A rechargeable 8 volt lithium ion polymer battery such as, for example, (model LIPBA-300-8, rated at 300 mAh/8 v) available from OPRA-TECH Engineering in Warren, Ohio, U.S.A., may also be used.

Another exemplary line of pumps suitable for use in the present embodiments is available from the Oken Seiko Co., Ltd. in Tokyo, Japan. One particular pump model that can be used in the present embodiments includes model S02R6331, which can operate on between about 1.5 to about 3.0 volts. Consequently, such a pump may be powered by a small capacitor, 1-2, 1.5 v AAA disposable or rechargeable batteries, or any other acceptable standard batteries.

Yet another type of vacuum pumps suitable for use in the present embodiments includes the model SA0002005 manufactured by Dynaflo of Birdsboro, Pa., U.S.A. With the appropriate electronics and controls, these pumps have been found to work well and may be adequately powered by a single lithium ion battery. While several acceptable batteries may be used for this purpose, the LP561943A lithium ion battery manufactured by Sanyo GS has been found to be useful due to its small size and reliability. When using a lithium ion battery, a circuit can be incorporated to protect both the user and the battery from the potential effects of battery misuse. One suitable circuit includes the G7070 protection circuit module made by Nexcon Technology Company of Korea. The G7070 module can be small in size and can offer a comprehensive array of protection functions.

The pumps described herein having appropriate size and power for use in an embodiment of the present disclosure can include a diaphragm comprising of Ethylene Propylene Diene Monomer (EPDM) rubber. EPDM can be used as a diaphragm material because of its performance under a variety of conditions for long periods of time. Unfortunately, in some embodiments of the pump, the diaphragm can be exposed to a variety of substances that can adversely affect the material properties of the diaphragm. The exposure can result in premature failure, or otherwise adversely affect the performance of the pump. Some of the substances that can adversely affect an EPDM pump diaphragm include perspiration, exudate from a prosthetic liner (especially mineral oil), lubricants, and cleaning substances.

Certain elastomers, which are not commonly used in pump diaphragms, have been found to perform better under such conditions than EPDM. These elastomers include, for example: silicone, fluorocarbon elastomers, florosilicones, neoprene, and Hydrogenated Nitrile Butadiene Rubber (HNBR). While these elastomers might not provide the same level of long term performance as EPDM in some applications, the elastomers can provide an improvement in useful life with respect to the conditions relevant to the present embodiments. Therefore, these alternative elastomers can also replace EPDM in other components of a vacuum system such as, for example, a check valve, and components exposed to the same or similar substances as the diaphragm.

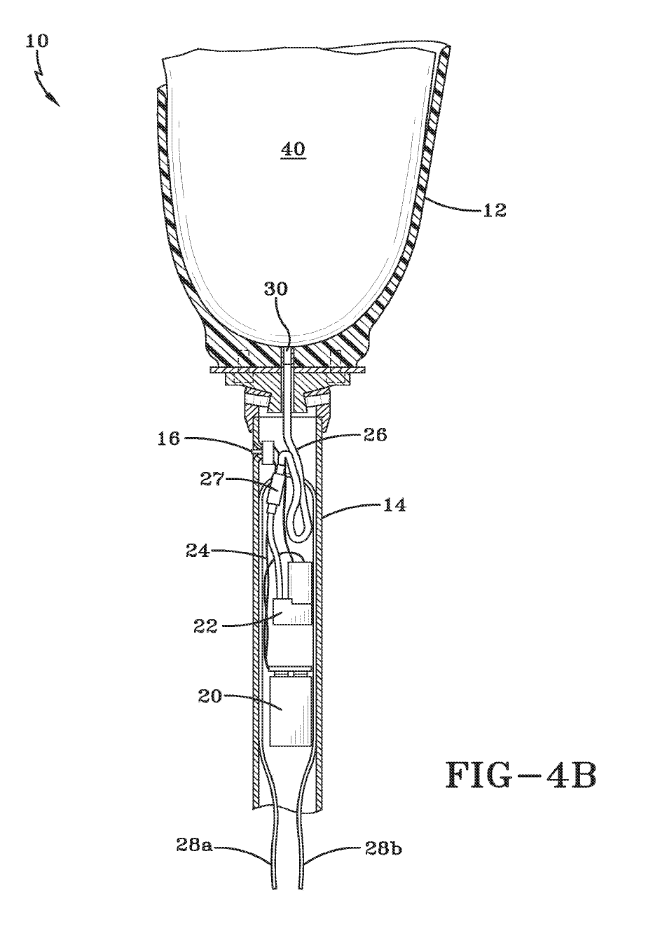

Therefore, it can be seen that electrically-powered vacuum pumps can be provided with a size and weight that permits their installation on or within the pylon 14, a housing, or another component of a prosthesis without substantially increasing the effort and drain on the patient using the prosthesis. Similarly, such pumps can be incorporated into a portable inflation pump such as, for example, embodiments illustrated in FIG. 5 below.

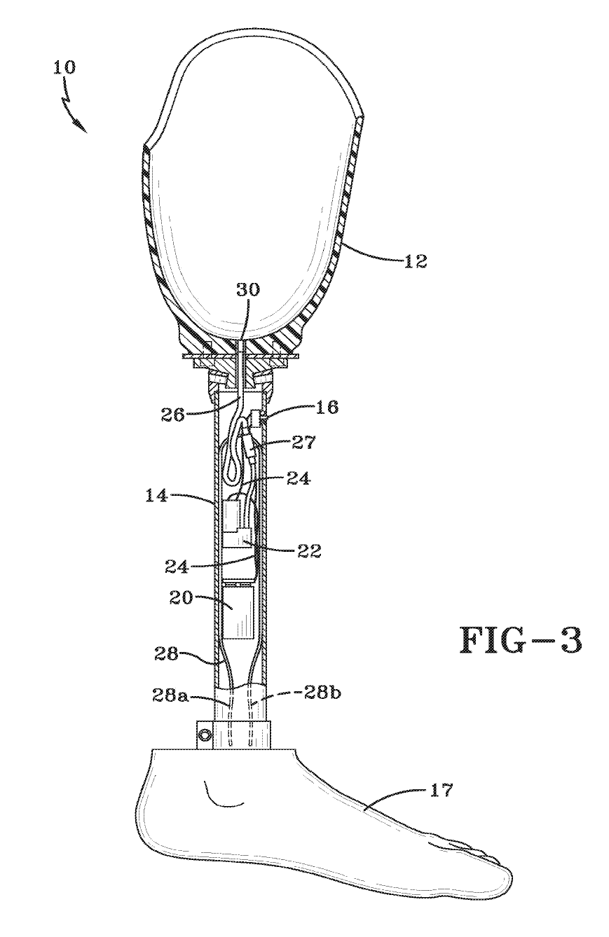

Referring now to FIG. 3, the prosthetic device 10 components can be inserted into the pylon 14. The ribbon 28 can form a loop surrounding the power source 20 and the vacuum pump 22. The power source 20 and the vacuum pump 22 may be withdrawn from the pylon 14 by pulling at the ends 28a and 28b of ribbon 28, which can extend to the bottom end of pylon 14. An electrical circuit can be formed by the electrical connections 24, the positive and negative contacts of the power source 20 and the positive and negative terminals of vacuum pump 22. One electrical connection can directly connect one terminal of the power source 20 to one terminal of vacuum pump 22, while further electrical connections can connect the other terminal of the power source 20 to the other terminal of vacuum pump 22 via electrical switch 16. Thus, by closing electrical switch 16, electrical power can be supplied to the vacuum pump 22, causing the vacuum pump 22 to operate and evacuate the socket 12.

A user of a prosthetic device as thus described can readily create elevated vacuum to any level desired, at least to the limits of vacuum that can be drawn by the vacuum pump 22. No particular vacuum level is required or contemplated by this particular embodiment, as individual patients may have specific preferences and physical and/or physiological needs that dictate the level of vacuum drawn. The described exemplary vacuum pumps can each have a flow rate sufficient to evacuate a typical socket to the desired vacuum level within about 30 seconds of vacuum pump operation. Some users can require very little vacuum within the socket 12, whereas others can desire a higher level of vacuum and may, therefore, operate the vacuum pump for a longer period of time. For example, certain levels of vacuum may be desirable due to their potential to reduce the risk of ulceration and improve vascular flow. Furthermore, the amputee may readily re-apply vacuum using the pump as described above as needed.

Referring collectively to FIG. 2 and FIG. 3, the vacuum line 26 can connect the vacuum pump 22 to a vacuum orifice 30 located in the socket 12 so that the socket may be evacuated by operation of the vacuum pump. Air drawn through the vacuum line 26 can be expelled via an outlet port 22b (FIG. 2) on vacuum pump 22 into the interior of the pylon 14. Air expelled into the pylon 14 can be vented to the atmosphere, as the interior of the pylon 14 can be selectively sealed from the atmosphere.

The vacuum line 26 can include a check valve 27 for permitting airflow through the vacuum tube 26 to the vacuum pump 22 but preventing reverse airflow from the vacuum pump through the vacuum tube and into the socket 12. The check-valve 27 may be a duckbill-valve or another known type of one-way valve.

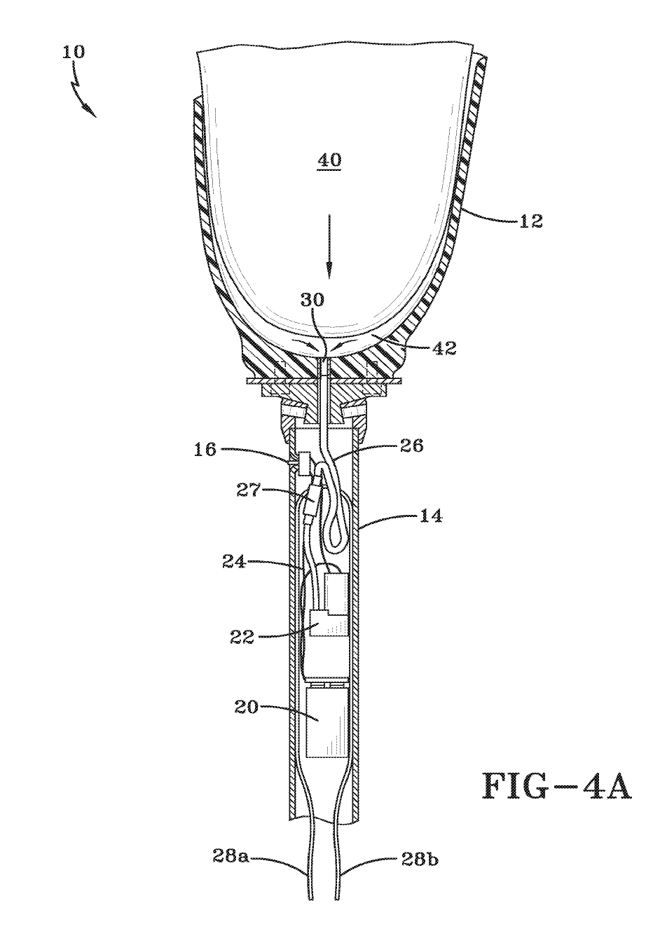

Referring now to FIG. 4A, the prosthetic device 10 can be used in connection with a patient's residual limb. A patient's residual limb 40, which can have a liner donned thereon, can be inserted into the socket 12. The socket 12 can be left with a cavity 42 filled with air. In an application wherein a liner without an outer fabric covering is used, an air wick sheath such as, for example, a fabric can be used to prevent the urethane, silicone, or thermoplastic liner from sealing the vacuum orifice and thus limiting the vacuum to the opening of the orifice only. Use of an air wick sheath over such a liner can allow air to be evacuated over a larger area of the residual limb. In applications wherein a fabric covered liner, such as one of the Alpha® family of liners available from The Ohio Willow Wood Company in Mt. Sterling, Ohio, U.S.A., is used, the use of an air wick sheath is unnecessary.

With the liner-covered residual limb inserted into the socket 12, the patient can depress the actuator button 16, activating the vacuum pump 22 and causing air from the cavity 42 to be drawn through the vacuum tube 26 and the check valve 27 to the vacuum pump 22. In turn, the air can be expelled into the interior of the pylon 14. The resulting vacuum in the cavity 42 can draw the residual limb 40 into tight coupling with the interior of the socket 12, and can permit use of the prosthetic device 10 for various ambulatory activities. The vacuum induced coupling between the residual limb 40 and the interior of the socket 12 (FIG. 4B).

Referring now to FIG. 5, according to embodiments described herein, the vacuum pump 22 or the power source 20 can be removed from the pylon 14. The pylon 14 can be configured to contain only the vacuum line 26, which can be coupled to the interior of the socket 12. The vacuum line 26 can connect to a vacuum orifice coupler 50/52, which can include two parts. A first part of the coupler 50/52 can include a check valve 50 that permits airflow from the socket 12 through the vacuum line 26, but blocks reverse airflow from the exterior environment into the vacuum line and socket. As shown, the coupler 50/52 may also include an orifice 52 for receiving a vacuum line from an external portable vacuum pump 56.

A portable evacuation device 56 can include a vacuum line 54 with a coupler 55 on the end thereof for connection to the vacuum orifice coupler 52. The interior of the portable evacuation device 56 can include a power source 60, such as a capacitor or battery, a vacuum pump 62, and a control switch 66. The power source 60 can be electrically connected to the vacuum pump 62 via electrical connections similar or identical to those described above with reference to FIGS. 2-4B, and the vacuum line 54 can be connected to the inlet port of the vacuum pump 62. The portable evacuation device can thus be used to draw air from the socket 12 by connecting the coupler 55 to the coupler 52, then actuating switch 66 to activate vacuum pump 62 and draw the air through the vacuum line 54.

Accordingly, the weight of the power source 60 and the vacuum pump 62, can be removed from the prosthesis. Also, a patient with a relatively long residual limb and, therefore, a short pylon 14, may not have sufficient volume in the pylon to enclose the motor and/or power source therein. Similarly, above-knee amputees may not have enough room to incorporate a vacuum system between a prosthetic knee coupler and the end of the user's socket. In such cases, a portable evacuation device may be utilized to provide a portable vacuum source for the amputee.

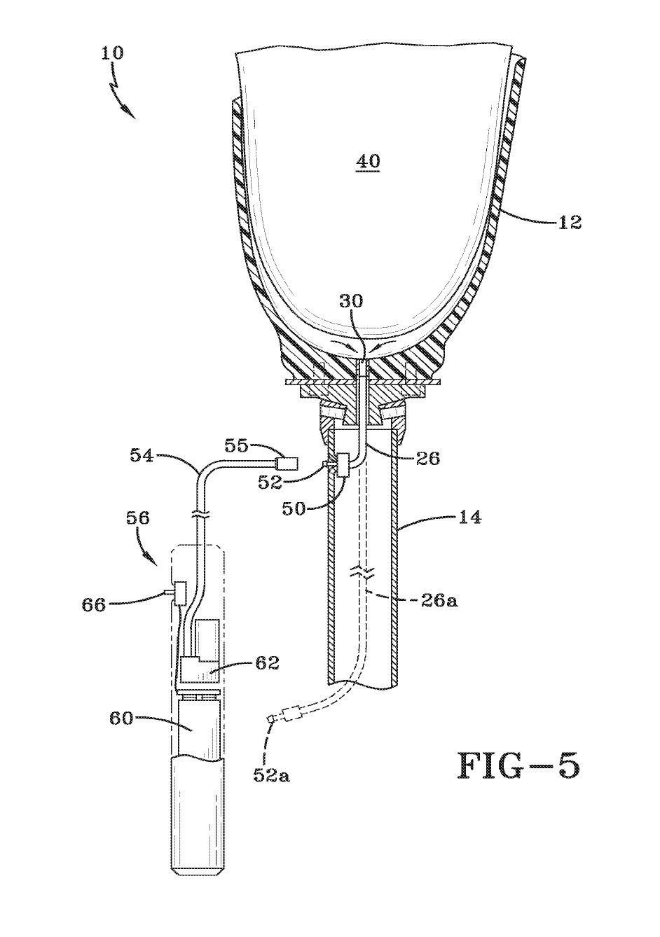

Referring now to FIG. 6, a vacuum pump 22 and power source 20 are again installed to a pylon 14. In some embodiments, the vacuum pump 22 and the power source 20 can be installed into a special sleeve 68 prior to insertion into the pylon 14. The sleeve 68 can be formed from a thin and lightweight material that may substantially conform to the shape of the pylon interior. In some embodiments, the sleeve 68 can consist of a thin plastic tube, although the use of other materials is possible. One or both ends of the sleeve 68 may be open, or the end(s) may be closed but for small access openings required for vacuum lines or electrical wiring.

The vacuum pump 22 and power source 20 may be retained within the sleeve 68 by a tight fit between the components and the interior of the sleeve. Alternatively, the sleeve interior may be provided with a special geometry designed to mate with and retain the vacuum pump 22 and/or power source 20.

With the vacuum pump 22 and power source 20 installed in the sleeve 68, the housing can be inserted into the pylon 14. Retention of the sleeve 68 within the pylon 14 can be achieved by a tight fit between the sleeve and the pylon interior or, a retention means may be provided. Such a retention means can include, for example, a pin, fastener, tab or other retainer that releasably affixes the sleeve 68 to the pylon 14. Various types of releasable adhesive, such as one or more pieces of double-stick tape or Velcro® may also be used for this purpose. Alternatively or additionally, retention of the sleeve 68 can be accomplished by means of a detent 70. More specifically, when the sleeve 68 is properly inserted into the pylon 14, a projection 72 located on the exterior of the housing can engage a hole or aperture 74 provided in the wall of the pylon. The interaction between the projection 72 and the aperture 74 can retain the sleeve 68 during normal use of an associated prosthesis, while also allowing for disengagement and deliberate removal of the sleeve if desired. The sleeve 68 may be used in any embodiment wherein a vacuum pump and power source are installed within a pylon or other hollow prosthetic component.

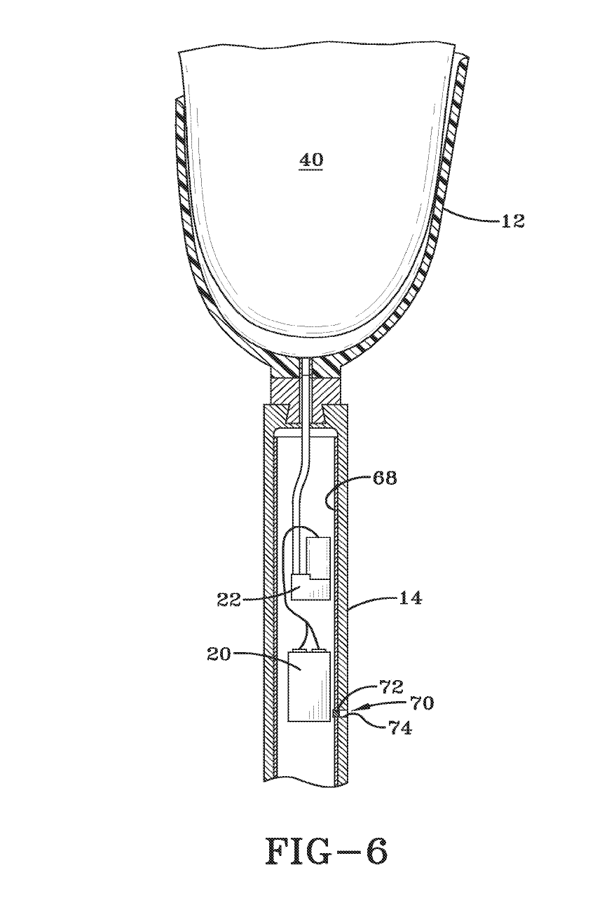

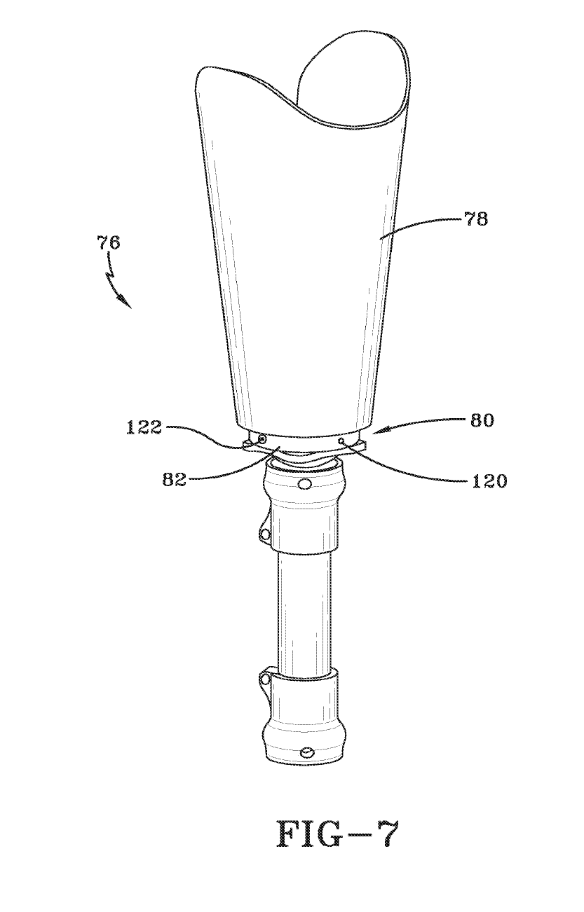

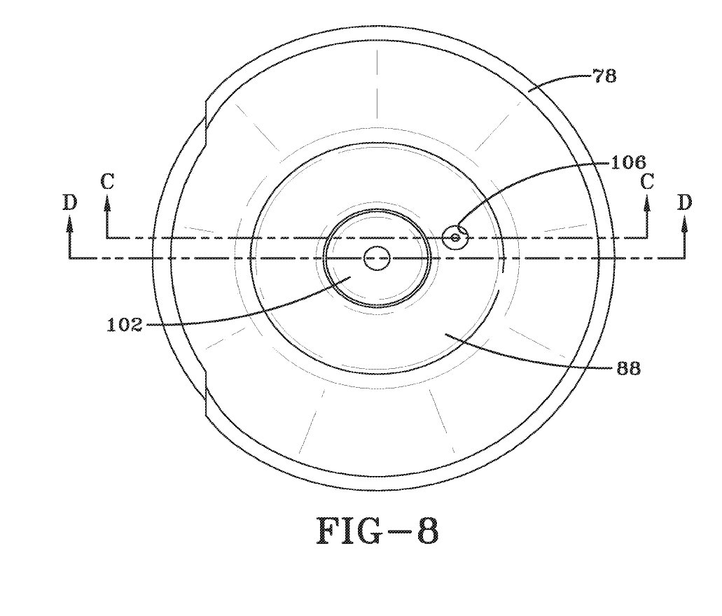

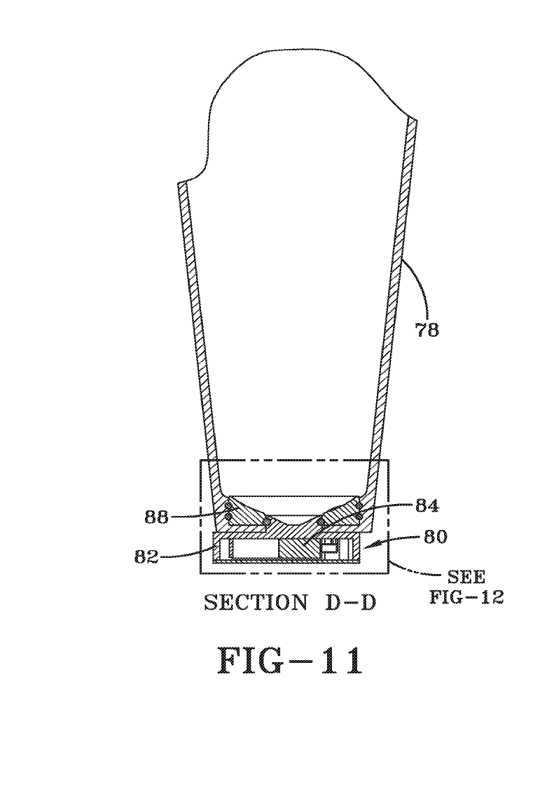

Referring collectively to FIGS. 7-12, a prosthetic limb 76 can include an evacuation device 80 having a vacuum pump 84 and power source 86 located in a housing 82. The housing 82 can be designed to mate with a universal distal adapter 88 affixed to or integrated into a prosthetic socket 78. Such a distal adapter 88 can be substantially located in the distal end of the prosthetic socket 78 (FIGS. 8-12), and may employ the four-hole attachment pattern common to the prosthetics industry.

A proximal (mounting) face 88a of the distal adapter 88 can reside interior to the socket 78 and can be concave, to better receive the distal end of the residual limb. The distal adapter 88 can have an aperture 90 passing axially therethrough. The aperture 90 can allow for the passage of various suspension components such as, for example, locking pins and lanyards, and also receives a portion of the evacuation device housing 82 when the evacuation device 80 is used. Suspension devices associated with such suspension components can be designed to mate with the distal adapter 88 in the same manner as the evacuation device 80, and these devices may be made to be interchangeable.

In embodiments, wherein a suction seal is desired, the distal adapter 88 can be equipped with one or more o-rings 92 or similar sealing elements that traverse its periphery and assist with providing an air-tight seal between the outer surface of the distal adapter 88 and the interior of the socket 78. Other sealing means may also be employed.

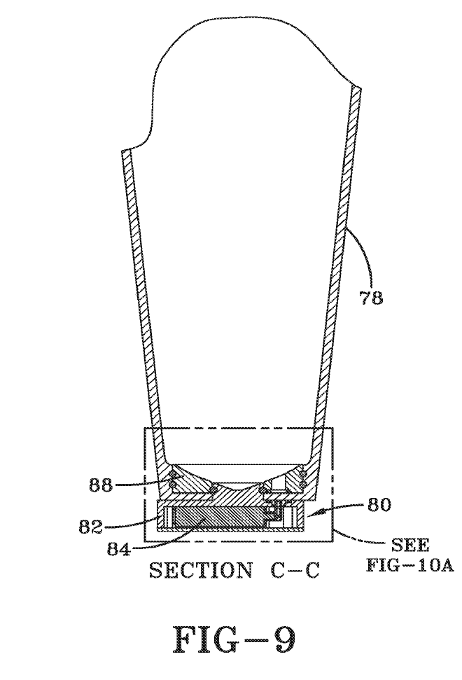

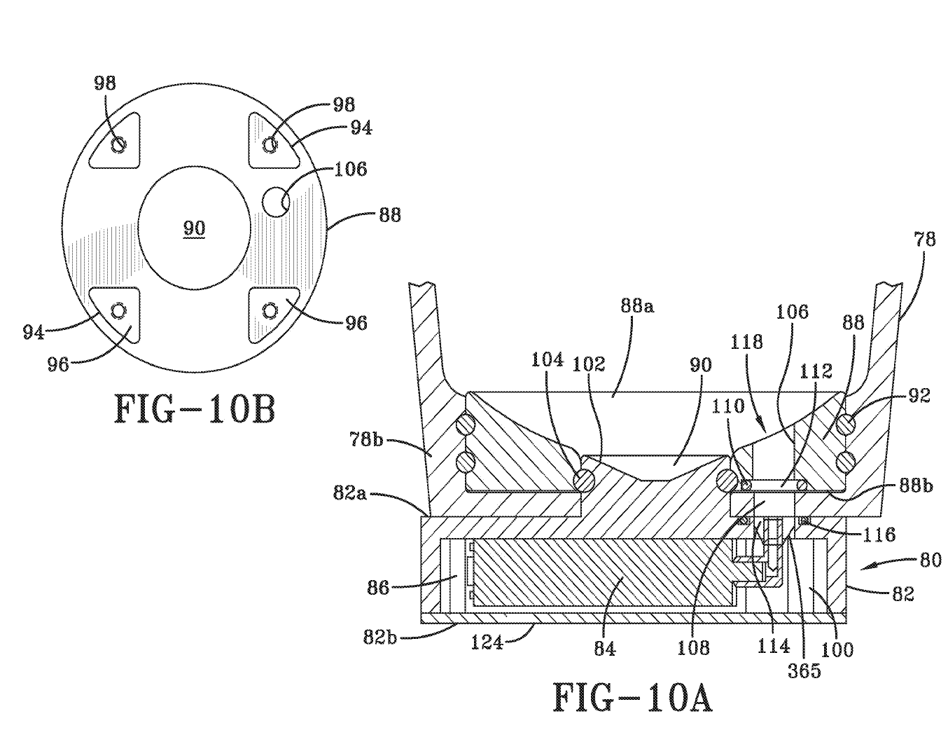

Referring to FIG. 10B, a number of mounting projections 94, each having a flat mounting surface 96, can extend downward from a bottom (connecting) face 88b of the distal adapter 88 and can be exposed along the bottom of the distal end 78b of the socket 78. This can be achieved during lamination of the socket 78 by employing a temporary cover plate to protect the mounting surfaces 96 and the aperture 90, while simultaneously allowing socket material to fill the channels formed between the mounting projections 94. As a result, a substantially flat mounting area can be provided at the distal end 78b of the socket 78, and can be provided with an aperture that connects the interior of the socket to the atmosphere via the aperture 90 in the distal adapter 88. In other embodiments, a distal adapter can include a single uniform mounting surface that is exposed along the distal end 78b of the socket 78 in place of mounting projections. It should be noted that any of embodiment of the distal adapter can be used with thermoplastic sockets to create either diagnostic or definitive sockets. The distal adapter 88 can be used in a thermoplastic diagnostic socket as well as a definitive socket—whether the definitive socket is laminated or thermoplastic.

Each mounting surface 96 can have a threaded mounting hole 98 for receiving a like-threaded fastener. The mounting surfaces 96 can mate with the proximal (mounting) side 82a of the evacuation device housing 82 when the evacuation device 80 is affixed to the distal end of the socket 78. The housing 82 can have a number of thru-holes 100 that are arranged to align with the mounting holes 98 located in the mounting surfaces 96 of the distal adapter 88. Fasteners may be passed through the thru-holes 100 in the housing 82 and threaded into the distal adapter mounting holes 98 to secure the evacuation device 80 to the distal end of the socket 78.

Referring collectively to FIGS. 7-12, various prosthetic components may be affixed to the distal (connecting) side 82b of the evacuation device housing 82 by the same fasteners. These prosthetic components may include, for example, pyramid adapters, Symes adapters, prosthetic ankles, prosthetic feet, prosthetic knees, and other components forming the remainder of a prosthesis.

According to the embodiments described herein, a sealing extension 102 can project upward from the mounting face 82a of the evacuation device housing 82 through the aperture 90 in the distal end 78b of the socket 78 and into the aperture 90 in the distal adapter 88. The sealing extension 102 can carry an o-ring 104 that acts to seal the aperture 90 in the distal adapter 88.

With the above-described construction, the distal end 78b of the socket 78 can be substantially air tight. As such, mating vacuum passages 106, 108 can extend through the distal adapter 88 and the distal end 78b of the socket 78. The vacuum passage 108 in the socket 78 may be created during lamination by means of a projection on the cover plate used to expose the mounting faces 96 of the mounting projections 94. Alternatively, the vacuum passage 108 may be bored through the distal end 78b of the socket 78 after lamination thereof. The interface of the vacuum passages 106, 108 may be further sealed with an o-ring 110 if desired. Such an o-ring 110 may be installed into a recess or counterbore 112 in the distal adapter 88.

An evacuation device vacuum passage 114 can extend from the vacuum pump 84 through the mounting surface 82a of the evacuation device housing 82. The evacuation device vacuum passage 114 can be aligned and can mate with the vacuum passages 108, 106 in the socket 108 and distal adapter 106 when the evacuation device 80 is properly mounted to the distal end 78b of the socket 78. An o-ring 116 or similar sealing element may be located in the mounting face 82a of the evacuation device housing 82 and around the evacuation device vacuum passage 114 to ensure a good seal. The connected vacuum passages 106, 108, 114 can operate as one continuous vacuum passageway 118 that allows the vacuum pump 84 of the evacuation device 80 to evacuate air from the interior of the socket 78. A one way valve may be placed in any of the vacuum passages 106, 108, 114 to ensure that air cannot flow into the socket 78.

Air evacuated from the socket may be discharged by the vacuum pump 84 through an exhaust port 120. The exhaust port 120 may reside at various locations in the housing 82. The evacuated air may be discharged directly to the atmosphere, or into another prosthetic component, such as a pylon, where it can thereafter leak to the atmosphere. A one-way valve and/or muffler can be associated with the exhaust port 120.

Alternatively, an evacuation device vacuum passage may pass from a vacuum pump through the sealing extension 102, instead of through the mounting face 82a of the housing 82. Thus, communication with the socket interior can occur through the aperture 90 in the distal adapter 88 and, therefore, the distal adapter vacuum passage 106 and socket vacuum passage 108 can be eliminated or plugged.

The evacuation device 80 can include an actuator button 122 that protrudes through the housing 82 for easy access by the user. Other actuating means may also be used, some of which are described in more detail below.

Access to the vacuum pump 84, power source 86 and/or other components located within the evacuation device 80 may be accomplished through one or more access holes or panels (not shown) located in a side(s) of the evacuation device housing 82. Alternatively, the connecting face 82b of the evacuation device housing 82 may comprise a removable plate 124 that can be detached as needed to provide access to the vacuum pump 84, power source 86, and/or other components located within the evacuation device housing 82 (e.g., a microprocessor, radio, vacuum sensor, pushbutton switch, check valve, or filter). The evacuation device 80 can be a structural part of the prosthesis, can contain electronic components and, can contain a radio. In some embodiments, in addition to having sufficient strength, the vacuum device can be water resistant, or waterproof and can be configured to not interfere with radio transmissions.

It should be noted that embodiments described herein can include the use of the universal distal adapter 88. As mentioned briefly above, such a distal adapter can allow for the interchangeability of various suspension devices, such as the evacuation device, a pin lock device, or a locking lanyard device. Each such device can employ the same hole pattern so as to properly mate with the distal adapter 88. The aperture 90 in the distal adapter can be sized to allow the passage of a suspension component (e.g., a locking pin or lanyard), but can also be sealed (as described above) when suction suspension is employed.

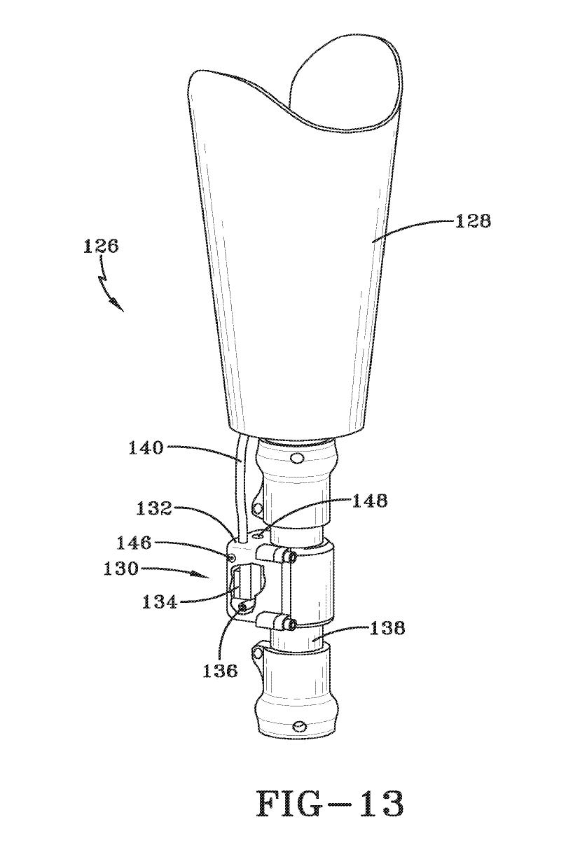

Referring now to FIG. 13, a prosthetic limb 126 can include an evacuation device 130 for evacuating a prosthetic socket 128. The evacuation device 130 can include a housing 132 containing at least a vacuum pump 134 and power source 136. The housing 132 can be designed to fit around a prosthetic pylon 138. The housing 132 may have two halves that can be fastened together around the pylon 138. In a variation of the evacuation device 130, the housing 132 may be of substantially one-piece construction having a passageway therethrough for receiving a pylon. The housing 132 may be retained on the pylon through an interference fit, or by a clamping means, for example.

Although shown to be substantially rectangular in cross-section in FIG. 13, the housing 132 may be contoured. For example, the housing 132 may be contoured in a similar fashion to a human calf, or some other appropriate or pleasing shape.

The vacuum pump 134 may be connected to the interior of the socket 128 by a vacuum line 140 that runs through the pylon 138. Accordingly, an aperture can be provided through the pylon for passage of the vacuum line. Alternatively, the vacuum line 140 may extend from the vacuum pump 134, through the housing 132 and distal end of the socket 128, and into the socket interior. As yet another alternative, a vacuum line 140 may extend from the vacuum pump 134, through the housing 132, and to a manifold (such as the manifold 290 described in detail below), which manifold provides for communication with the socket interior so that air can be drawn therefrom.

An actuator button 146 may extend through the housing for easy access by the user. Other actuating means may also be provided as described in more detail below.

Air evacuated from the socket may be discharged by the vacuum pump 134 through an exhaust port 148. The exhaust port 148 may reside at various locations in the housing 132. A one-way valve and/or muffler can be associated with the exhaust port 148.

Access to the vacuum pump 134, power source 136 and/or other components located within the evacuation device housing 132 may be accomplished by separating the halves of the evacuation device housing.

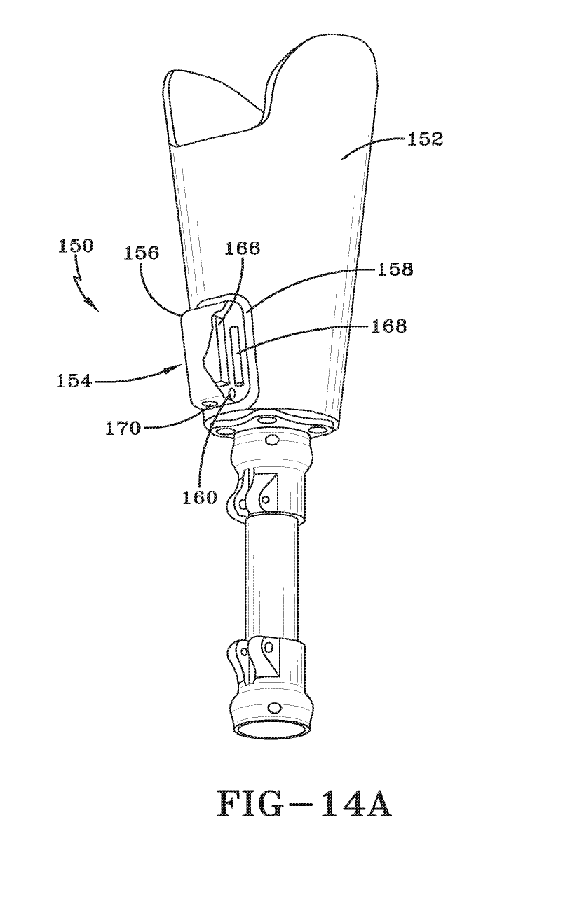

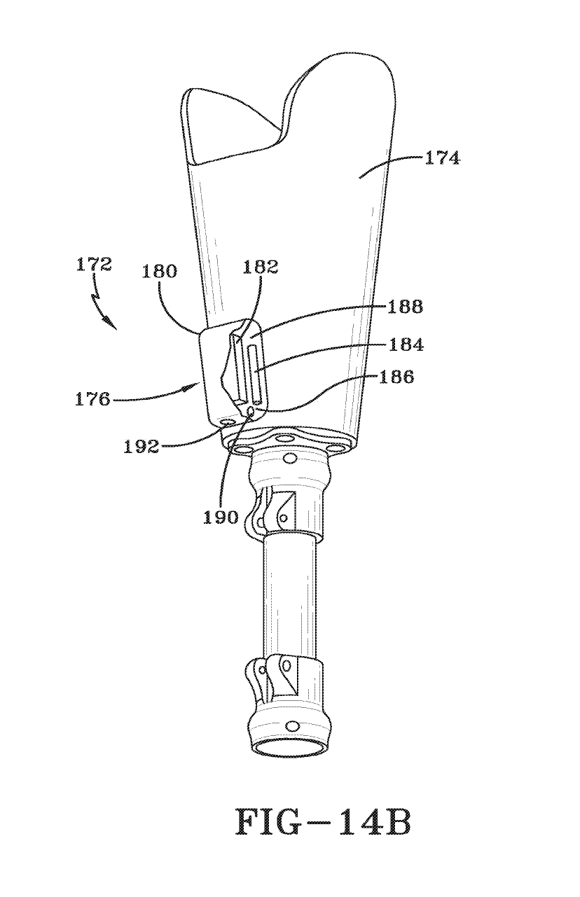

Referring now to FIG. 14A, an evacuation device 154 can include at least a vacuum pump 166 and power source 168 contained within a housing 156 that is attached to a side wall of a socket 152 of a prosthetic limb 150. In one embodiment, the housing 156 can be affixed to a mounting adapter 158 that is built directly into the socket 152, such as during the lamination thereof.

A vacuum passage 160 may extend through the mounting adapter 158 and socket sidewall, and into to the interior of the socket 152. Air may be evacuated from the socket interior by drawing it through the vacuum passage 160 using the vacuum pump 166.

Air evacuated from the socket 152 may be discharged by the vacuum pump 166 through an exhaust port 170. The exhaust port 170 may reside at various locations in the housing 156 or in the mounting adapter 158. When a manifold is used, an exhaust port may be located therein. A one-way valve and/or muffler can be associated with the exhaust port 170 regardless of its location.

Referring now to FIG. 14B, a prosthetic limb 172 can be provided with an evacuation device 176. The evacuation device 176 can include a vacuum pump 182 and power source 184 residing within a housing 180 that is integral to a side wall of a prosthetic socket 174. The housing 180 can protrude from the side wall of the socket 174 and can form a chamber 186 within which the vacuum pump 182 and power source 184 can be retained. The housing 180 may be a separate component that is laminated or otherwise bonded to the socket 174 after the socket is formed. In one embodiment, the housing 180 can be formed along with the socket 174.

The vacuum pump 182 and power source 184 may be permanently sealed within the chamber 186. Alternatively, a removable interior cover 188 may be provided to ensure that the vacuum pump 182, power source 184, and any other associated components remain within the chamber 186, while allowing access thereto when required.

A vacuum passage 190 or vacuum line may extend into the interior of the socket 174. When an interior cover 188 is present, the vacuum passage 190 or a vacuum line may extend therethrough. Air can be evacuated from the socket interior by the vacuum pump 182 via the vacuum passage 190.

Air evacuated from the socket may be discharged by the vacuum pump 182 through an exhaust port 192. The exhaust port 192 may reside at various locations in the housing 180. A one-way valve and/or muffler can be associated with the exhaust port 192.

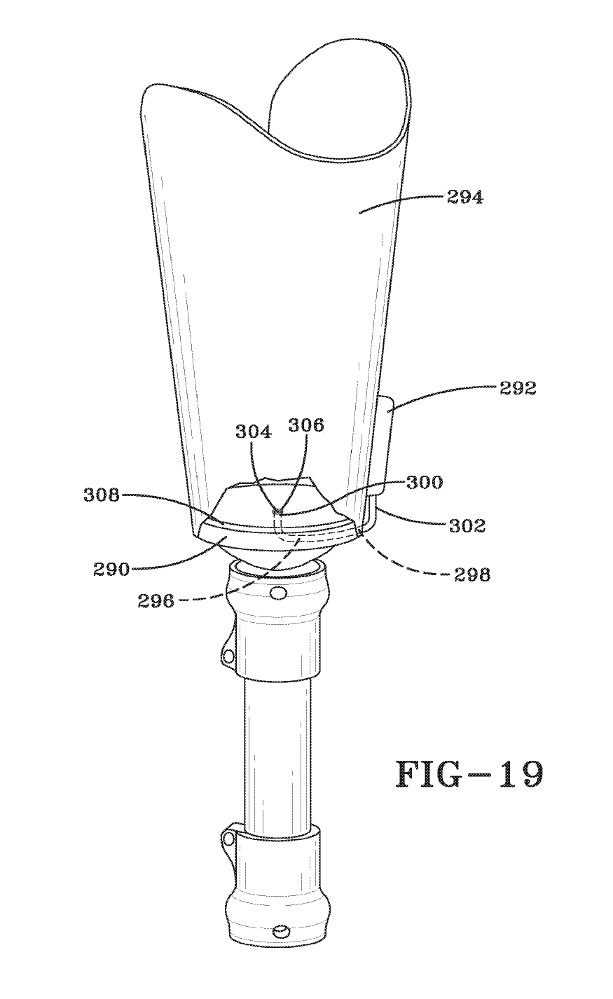

Referring collectively to FIGS. 14A and 14B, a vacuum line may run from the vacuum pump 166, 182, through the housing 156, 180, and to a manifold connected to the socket 152, 174, such as, for example, the manifold 290 (FIG. 19). The manifold can provide access to the interior of the socket 152, 174, such that air may be drawn therefrom by the vacuum pump 166, 182. In some embodiments, a vacuum line may run from the vacuum pump 166, 182, through the housing 156, 180, and to a vacuum passage located more remotely from the evacuation device, such as on the bottom surface of the socket.

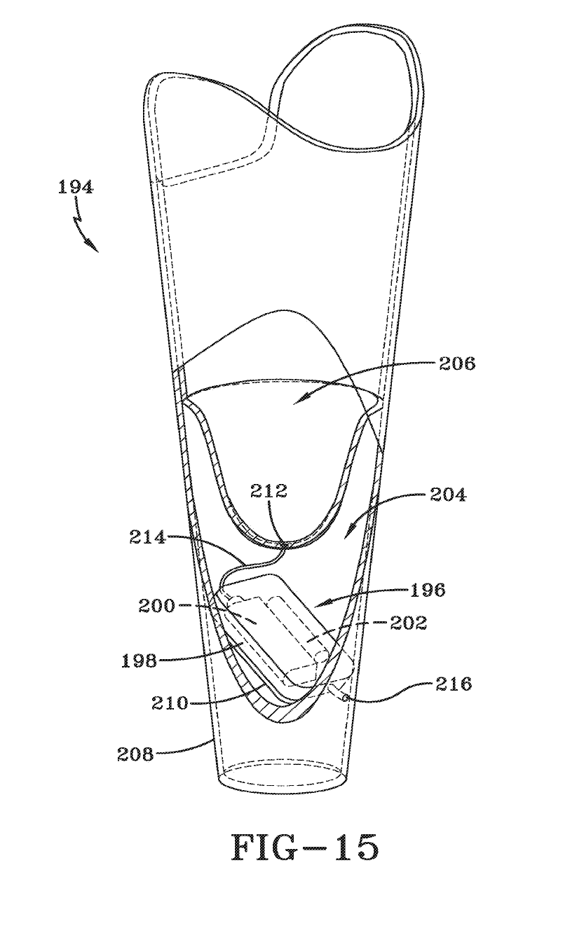

Referring now to FIG. 15, an evacuation device 196 can include a vacuum pump 200 and power source 202 located in a housing 198. The housing 198 can be positioned within an exoskeletal prosthetic device 194. More specifically, the housing 198 can be located within a cavity 204 between a socket portion 206 and distal end 208 of the exoskeletal prosthetic device 194. Such an exoskeletal prosthetic device 194 may account for a majority of a prosthetic leg or prosthetic arm, for example.

The evacuation device 196 may be secured within the exoskeletal prosthetic device 194 in any number of ways. For example, when the evacuation device 196 includes a housing 198, straps, clips, tabs, releasable adhesives, Velcro®, and any number of other types of retainers may be secured to the interior of the exoskeletal prosthetic device 194 and used to engage and retain the housing. Such retainers can also be provided to individually secure the vacuum pump 200 and power source 202 within the exoskeletal prosthetic device 194 in embodiments wherein no evacuation device housing is used.

Alternatively or additionally, a mounting pad, plate or other such structure may be fabricated or otherwise secured within the cavity 204 of the exoskeletal prosthetic device 194 to provide an attaching surface 210 for the housing 198. The housing 198 may be secured to the attaching surface 210 using any of the retainers mentioned above, or by screws, double-sided tape, or any other known means.

A vacuum passage 212 can extend into the interior of the socket 206. A vacuum line 214 can connect the vacuum pump 200 of the evacuation device 196 to the socket interior via the vacuum passage 212. Air can be evacuated from the socket interior by the vacuum pump 200 using the vacuum passage 212 and vacuum line 214.

Air drawn from the socket interior may be discharged by the vacuum pump 200 directly to the atmosphere through an exhaust port 216 in the exoskeletal prosthetic device 194. Alternatively, air evacuated from the socket interior may be discharged into the cavity 204 in the exoskeletal prosthetic device 194. The air may thereafter leak to the atmosphere through one or more component interfaces or be released through the exhaust port 216, which may be manually or automatically actuated. The exhaust port 216 may include a one-way valve and/or muffler.

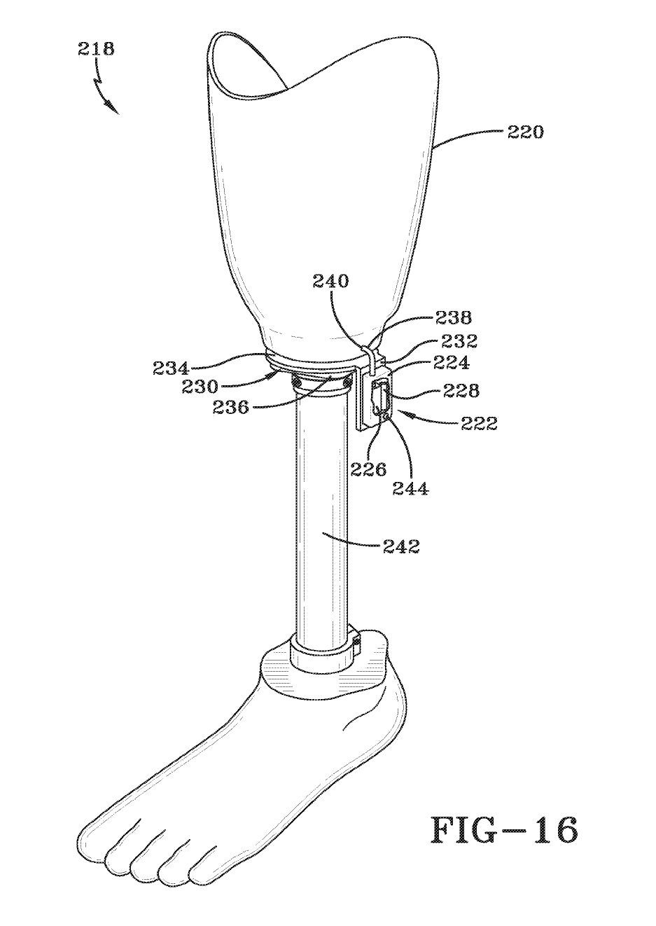

Referring now to FIG. 16, an evacuation device 222 can be affixed to a mounting plate 230 that is designed to be mounted between adjacent components of a prosthetic limb 218. In one embodiment, the evacuation device 222 can include a housing 224 that contains a vacuum pump 226 and power source 228. The housing 224 can be adapted for affixation to an attachment face 232 of the mounting plate 230. Alternatively, the vacuum pump 226 and power source 228 may be individually affixed to the attachment face 232 of the mounting plate 230 without a housing.

In one embodiment, the mounting plate 230 can be L-shaped, such that a mounting portion 234 thereof can be located between adjacent components of the prosthetic limb 218, while the attachment face 232 extends substantially parallel to the length of the prosthetic limb. The mounting plate 230 may be located between for example, without limitation, a prosthetic ankle and foot, or a prosthetic socket 220 and a pyramid adapter 236.

A vacuum line 238 may run from the vacuum pump 226, through the housing 224, if present, and into a vacuum passage 240 located in the socket 220 of the prosthetic limb 218. The vacuum line 238 may run between the vacuum pump 226 and socket 220 completely exterior to the prosthetic limb 218, as shown, or may be routed at least partially within the mounting portion 234, a pylon 242, and/or other components of the prosthetic limb. The portions of the vacuum line 238 that run exterior to the prosthetic limb 218 can be releasably secured to neighboring limb components.

Alternatively or additionally, a vacuum line may run from the vacuum pump 226 (through the housing 224, if present) to a manifold connected to the socket 220 such as, for example, the manifold 290 (FIG. 19). The manifold can provide access to the interior of the socket 220, such that air can be drawn therefrom. The manifold can be used with any of the above-described routings of the vacuum line 238.

Air evacuated from the socket 220 may be discharged to the atmosphere by the vacuum pump 226. The evacuated air may be discharged through an exhaust port 244, which may be located in/on the vacuum pump 226, or at various locations in the housing 224 (if present). When a manifold is used, an exhaust port may be located therein. A one-way valve and/or muffler can be associated with the exhaust port, regardless of its location.

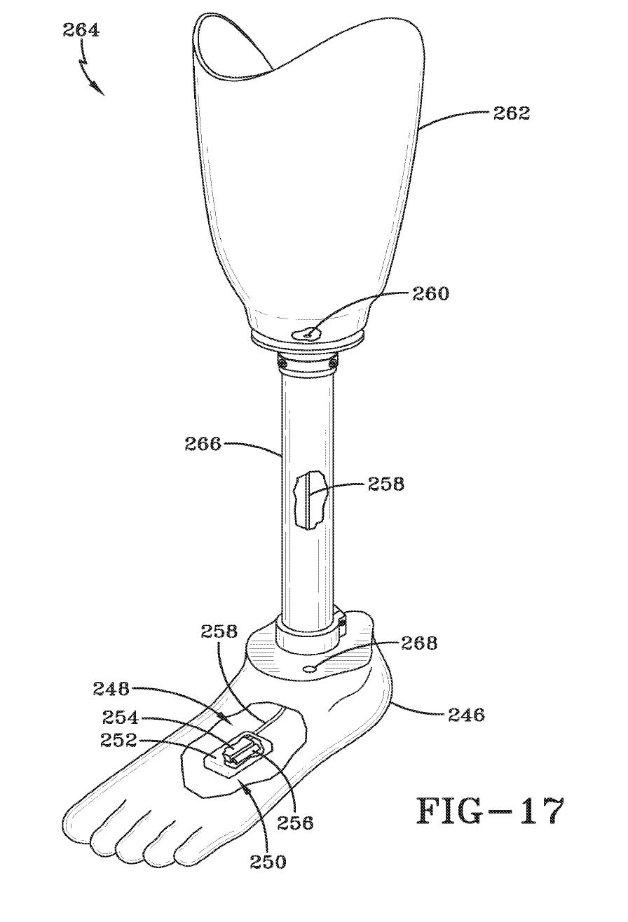

Referring now to FIG. 17, an evacuation device 250 can be located within a prosthetic foot 246, which may be a solid prosthetic foot or a hollow foot covering. For example, the evacuation device 250 may consist of a vacuum pump 254 and associated power source 256 that reside within a cavity 248 in the foot 246. In some embodiments, the evacuation device 250 can also include a housing 252 that contains the vacuum pump 254 and power source 256. The housing 252 can be located in the prosthetic foot cavity 248.

A vacuum line 258 may run from the vacuum pump 254, through the prosthetic foot 246, and into a vacuum passage 260 located in the socket 262 of the prosthetic limb 264. The vacuum line 258 may run between the vacuum pump 254 and socket 262 completely exterior to the prosthetic limb 264. Alternatively, the vacuum line 258 may be routed at least partially within a pylon 266 and/or other components of the prosthetic limb. For example, the vacuum line 258 can be routed from within the foot through a prosthetic ankle and pylon, and into the distal end of the socket. The portions of the vacuum line 258 that run exterior to the prosthetic limb 264 can be releasably secured to neighboring limb components.

Alternatively or additionally, the vacuum line 258 may run from the vacuum pump 254 to a manifold connected to the socket 262, such as, for example, the manifold 290 (FIG. 19). The manifold can provide access to the interior of the socket 262, such that air can be drawn therefrom. The manifold can be used with any of the above-described routings of the vacuum line 258.

Air evacuated from the socket by the vacuum pump 254 may be discharged to the atmosphere. In one embodiment, air is discharged through an exhaust port 268 located in the prosthetic foot 246. When a manifold is used, an exhaust port may be located therein. A one-way valve and/or muffler can be associated with the exhaust port, regardless of its location.

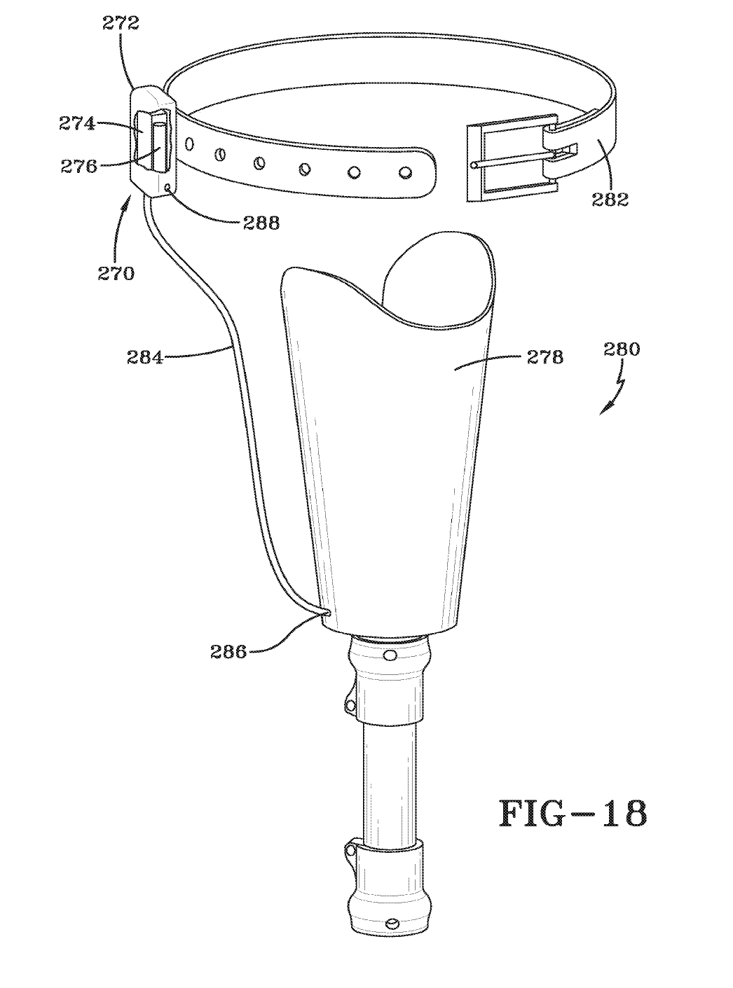

Referring now to FIG. 18, an evacuation device 270 can include a housing 272 containing at least a vacuum pump 274 and power source 276. The evacuation device 270 can be located on the user's person and can be provided to evacuate a socket 278 of a prosthetic limb 280.

The evacuation device 270 may clipped or otherwise attached to a user's belt 282. Alternatively, the evacuation device 270 may be placed in a pocket or temporarily attached to some other piece of a user's attire. The housing 272 may have an attachment mechanism such as a spring-loaded clip integral thereto. Alternatively or additionally, the housing 272 may fit into a sleeve or similar holder that acts to temporarily secure the evacuation device 270 to a user's attire. Such a holder may operate, for example, much like a clip-on cell phone holder.

A vacuum line 284 may run from the vacuum pump 274, through the housing 272, and into a vacuum passage 286 located in the socket 278 of the prosthetic limb 280. The vacuum line 284 may be routed at least partially under the user's clothing. Those portions of the vacuum line 284 that run exterior to the prosthetic limb 280 can be releasably secured to the prosthetic socket 278.

Alternatively or additionally, the vacuum line 284 may run from the vacuum pump 274 to a manifold connected to the socket 278, such as, for example, the manifold 290 (FIG. 19). The manifold can provide access to the interior of the socket 278, such that air can be drawn therefrom.

Air evacuated from the socket 278 by the vacuum pump 274 may be discharged to the atmosphere through an exhaust port 288 located in the housing 272. When a manifold is used, an exhaust port may be located therein. A one-way valve and/or muffler can be associated with the exhaust port, regardless of its location.

Referring now to FIG. 19, a manifold 290 can be provided to connect a vacuum source 292 to the interior of a prosthetic socket 294. The vacuum source 292 may be an evacuation device, a hand-operated vacuum pump, or some other vacuum device that can be connected to the manifold 290.

The manifold 290 can be associated with and attached to the distal end of the prosthetic socket 294. It should be realized, however, that the manifold 290 can be attached to other portions of the prosthetic socket 294, as long as the attached location permits access to the interior portion of the socket that is to be evacuated.

In some embodiments, a vacuum passageway 296 can extend through the manifold 290. One end 298 of the vacuum passageway 296 can be adapted to connect with or receive a vacuum line 302 that connects the manifold 290 to the vacuum source 292. The other end 300 of the vacuum passageway 296 can be adapted to align with a vacuum passage 304 that extends through the socket wall. The vacuum passage 304 can extend through the distal end of the socket 294, or can be located elsewhere in other embodiments. An o-ring 306 or other sealing element may be located at the interface of the vacuum passageway 296 and the vacuum passage 304 to help ensure a substantially air-tight seal.

The manifold 290 may be attached to the socket 294 in a number of different ways. For example, the manifold 290 may be laminated or otherwise bonded to the socket 294. Alternatively, the manifold 290 may be secured to a mounting plate 308 that has been integrated into the socket 294. The manifold 290 could also be affixed to the universal distal adapter 88 (FIGS. 8-12).

Using the vacuum source 292, air can be drawn from the socket interior through the manifold 290. The evacuated air may be discharged through an exhaust port associated with the vacuum source 292 or from some other location. As described above, a one-way valve and/or muffler can be associated with the exhaust port, regardless of its location.

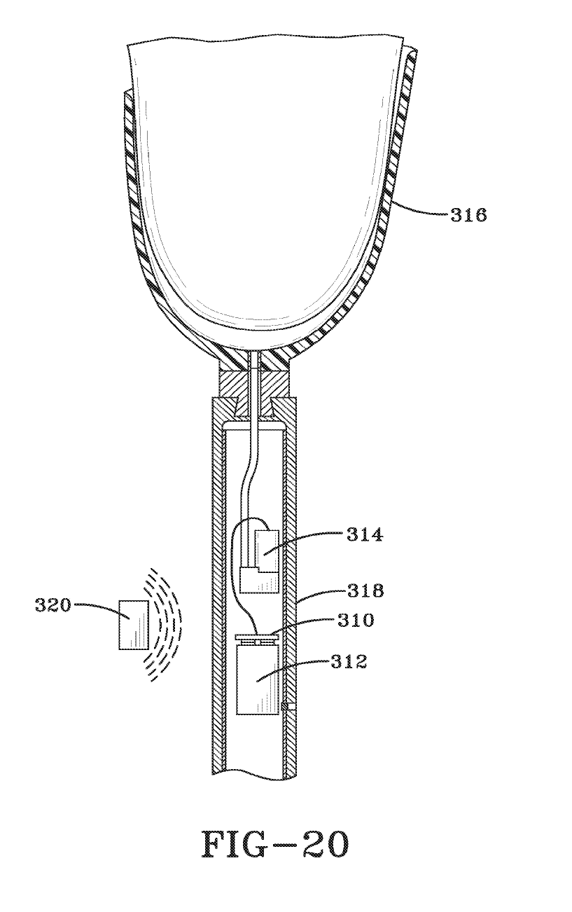

Referring now to FIG. 20, a magnetic switch 310 may be used in place of an actuator button or other vacuum pump actuator that requires direct contact by the user. The magnetic switch 310 can reside between a power source 312 and a motor of a vacuum pump 314. When actuated, the magnetic switch 310 can allow current to flow from the power source 312 to the motor, activating the vacuum pump 314 and initiating the evacuation process.

Unlike a protruding pushbutton or switch, however, actuation of the magnetic switch 310 can often take place through the material forming, for example, an evacuation device housing, a prosthetic socket 316, or a prosthetic pylon 318. More specifically, in some embodiments, a user can activate and deactivate the evacuation device by holding a small magnetic activator 320 in close proximity to the magnetic switch 310. Magnetic attraction between the magnetic activator 320 and the magnetic switch 310 can activate or deactivate the evacuation device as desired. Selective activation and deactivation can be accomplished, for example, by reversing the field of the magnetic activator 320 or by changing the location thereof with respect to the magnetic switch 310.

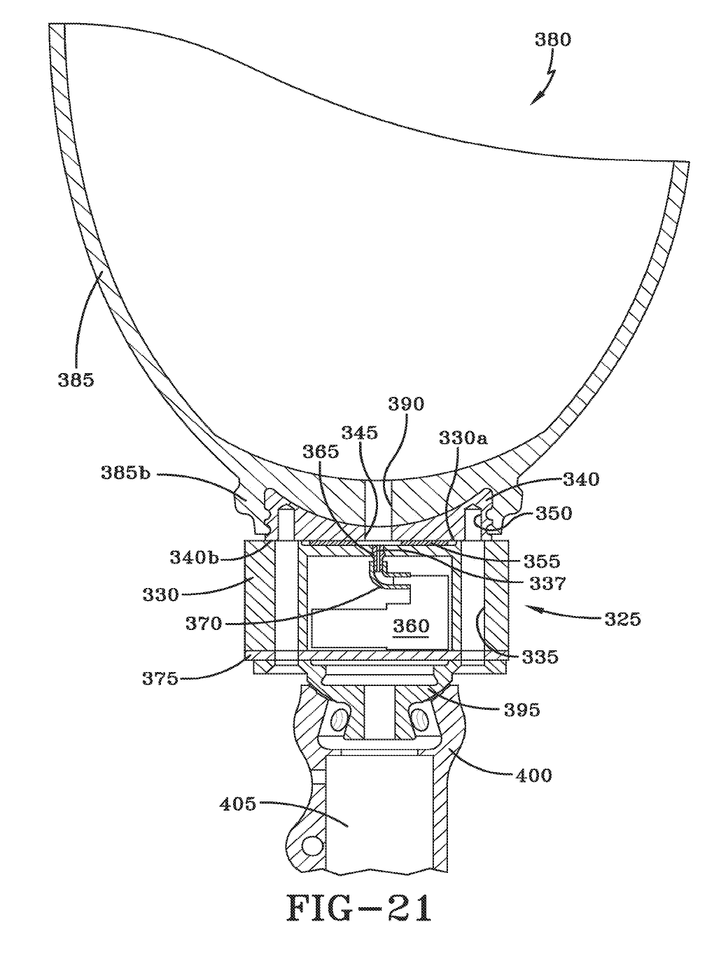

Referring collectively to FIGS. 7-12, and 21, An alternate version of an evacuation device 325 (FIG. 21) can be similar to the evacuation device 80 (FIGS. 7-12). In some embodiments, the upwardly projecting sealing extension 102 of the evacuation device 80 can be absent from the mounting face of the evacuation device 325. Likewise, the corresponding universal distal adapter 340 for receiving such an upwardly projecting sealing extension can be provided without an aperture.

The evacuation device 325 can include an evacuation device housing 330 adapted for mounting between the exterior distal end of a prosthetic socket 385 and a pylon 405, or other connecting component forming a portion of the remainder of the prosthetic limb 380. In some embodiments, the evacuation device 325 can be associated with a prosthetic leg and can be located between the distal end of the prosthetic socket 385 and a pyramid adapter 395. One end of the pyramid adapter 395 can be secured to a bottom surface 330b of the evacuation device housing 330 by fasteners that are used to secure the evacuation device 325 to the socket 385. The other end of the pyramid adapter 395 can be received by a pyramid receiver tube clamp 400 that connects a pylon 405 and the remainder of the prosthetic leg to the pyramid adapter and to the prosthetic socket.

The distal adapter 340 can be similar to the distal adapter 88. The distal adapter 340 can be installed into the distal end 385b of the prosthetic socket 385. In one embodiment, a bottom surface 340b of the distal adapter 340 can extend slightly from the exterior surface of the distal end 385b of the prosthetic socket 385. Alternatively, the bottom surface 340b of the distal adapter 340 may be flush with or slightly interior of the exterior surface of the distal end 385b of the prosthetic socket 385. The distal adapter 340 can include a thru-hole 345 that aligns with a thru-hole 390 passing through the distal end 385b of the socket 385. The thru-hole 390 in the socket 385 may be created during socket molding or afterward. Although the thru-holes 345, 390 are shown to be substantially axially located in FIG. 21, the thru-holes 345, 390 can be offset therefrom.

The evacuation device housing 330 can include a number of mounting holes 335 that align with the corresponding mounting holes 350 in the distal adapter 340, and allow the evacuation device 325 to be secured thereto. In one embodiment, the mounting holes 335 in the evacuation device housing 330 can be thru-holes and the mounting holes 345 in the distal adapter 340 can be threaded to receive like-threaded fasteners. In order to seal the top surface 330a of the evacuation device housing 330 to the exterior of the distal end 385 of the prosthetic socket 385, a gasket 355 can be located therebetween.

A vacuum pump 360 can be located within the evacuation device housing 330. The evacuation device housing 330 can include a vacuum passage (aperture) 337 that allows for communication between the vacuum pump 360 and the vacuum passageway formed by the aligned thru-holes 345, 390 in the universal adapter 340 and prosthetic socket 385.

The vacuum pump 360 can be connected to the vacuum passage (thru-hole) 345 in the universal adapter 340. In some embodiments, the connection can be made by inserting a barbed fitting 365 into the thru-hole 345 in the distal adapter 340 and connecting the vacuum pump 360 thereto with a piece of flexible tubing 370. Various other means of connecting the vacuum pump 360 to the thru-hole 345 in the distal adapter 345 may also be employed. For example, other types of fittings may be used, tubing may be inserted directly into the distal adapter thru-hole 345, or the vacuum pump 360 may be adapted for direct connection to the distal adapter thru-hole. In any event, the vacuum pump 360 can be operative to evacuate the interior of the prosthetic socket 385 by drawing air therefrom via the thru-holes 390, 345 in the distal end 385b of the prosthetic socket and in the distal adapter 340.

Any or all of the other features described above with respect to the evacuation device 80 may be possessed by the evacuation device 325. For example, and without limitation, a power source may be present within the evacuation device housing 330, and a lid or similar cover 375 may be provided thereon/therein to allow for access to the interior of the housing. Furthermore, the evacuation device housing 325 may employ the 4-hole mounting pattern of the evacuation device 80. The evacuation device housing 325 may be constructed of a material having sufficient strength and/or a material that does not interfere with radio signals. Evacuated air may be exhausted by the vacuum pump 360 in any manner previously described, or in another manner.

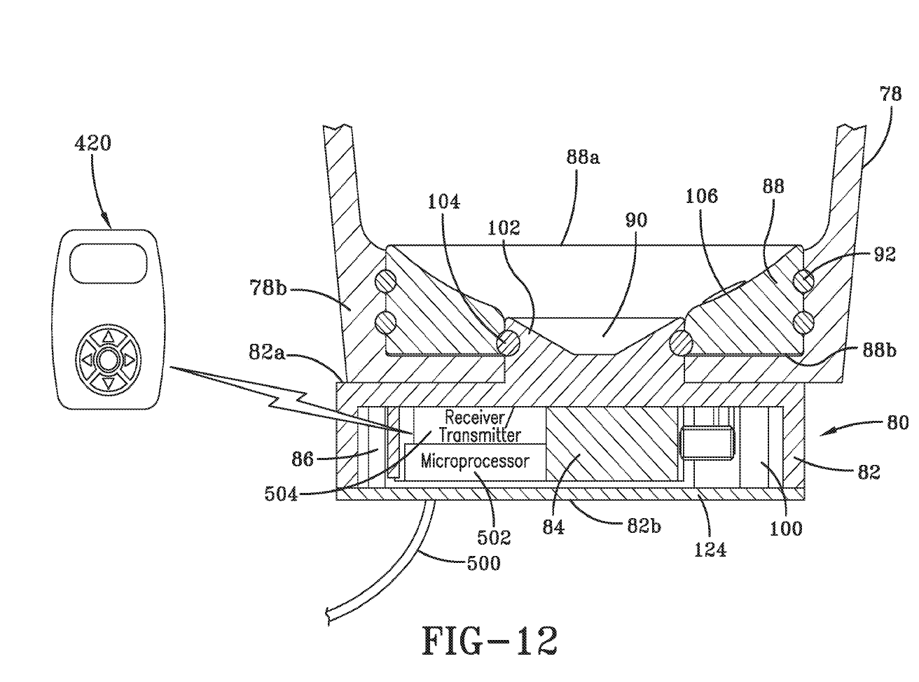

Referring again to FIG. 12, the vacuum pump 84 may be operated by various power sources, such as one or more batteries or capacitors. Accordingly, the power source 86 may need to be replaced. In some embodiments, the evacuation device 80 can be with easy access to the power source(s) 86 and/or, can employ a rechargeable power source(s).

When employing a rechargeable power source, recharging can be accomplished by either direct or inductive charging. In one embodiment of direct charging, the power source 86 can be connected to a plug-in charger that transfers electrical energy to the power source using the electrical circuitry of the evacuation device 80. For example, the evacuation 80 device may have a housing that includes a charging jack that is connected to the contacts of the power source 86. The power source 86 can be recharged by plugging an external charger into the charging jack.

In some embodiments, the evacuation device 80 can be configured mitigate uncertainty regarding the charge status of the power source 86. That is, a user may monitor or otherwise be informed of the charge status of the power source 86, and act accordingly if the charge level reaches a sufficiently low level.

In some embodiments, the evacuation device 80 can be provided with self-charging capabilities. For example, a small inductive generator may be located on the prosthetic limb and placed in electrical communication with the power source 86 of the evacuation device 80. Such a generator may be constructed and located on the prosthetic limb such that movement of the prosthetic limb during ambulation of the amputee will generate electric power by causing relative motion of coils within a magnetic field. Electrical energy produced by the generator can then be provided to the power source 86 of the evacuation device 80 to maintain the power source 86 in an acceptably charged state.

Other types of electric power generators may be employed for the same purpose. For example, an electro active polymer (EAP) generator could be associated with the prosthetic limb. EAP materials have evolved into a very viable alternative to other energy generation methods, and although EAP generators do have some limitations, these limitations are not insurmountable in a prosthetic device application. Alternatively, sufficient charging energy could also be generated using piezoelectric element generators. Piezoelectric elements can generate a voltage in response to applied mechanical stress and, therefore, can be caused to generate electrical energy by movement of a prosthetic limb to which they are attached.

Accordingly, the embodiments described herein can be provided with such self-charging capability. When so equipped, the evacuation device 80 can include any electrical circuitry necessary to receive electrical energy from the generator(s), and may also include circuitry and/or other elements to prevent over-charging of the power source(s).

With respect to the operational aspects of the evacuation devices of the present disclosure, basic through advanced versions are contemplated. More particularly, each embodiment of an evacuation device of the present disclosure may include a basic version that provides for manual operation only, an advanced version that is fully automatic, and one or more versions having operational features that fall somewhere therebetween.

The basic level can provide for manual operation. Manual operation can involve a user engaging an actuator that results in activation of a vacuum pump and evacuation of the prosthetic socket. The vacuum pump can continue to evacuate the socket until the user releases the actuator or the vacuum level reaches the maximum level that can be achieved by the pump. Thus, manual operation allows a user to select a vacuum level that best corresponds to his/her current activity level or desired comfort level. Vacuum can be periodically increased or decreased as desired by the user.

A semi-automatic mode can be achieved by adding certain types of sensors to the vacuum system, thereby requiring only minimal user interaction. For example, in one embodiment of semi-automatic operation, a pressure switch may be provided. The pressure switch can be configured to prevent the vacuum level from exceeding some level previously found to be uncomfortable or otherwise inappropriate for the user.

Alternatively or additionally, a pressure sensor can be configured to monitor vacuum level such as, for example, an absolute vacuum pressure sensor. The use of an absolute pressure sensor can result in an amputee experiencing significantly different inter-socket forces as a result of elevation changes. Such force differences can be exacerbated by extreme changes in elevation, such as between sea level and a high ground level (e.g., such as in Denver Colo.), or between a ground level altitude and the altitude achieved during airplane flight. Therefore, the pressure sensor can be configured to monitor gauge pressure, i.e., a pressure gauge can be exposed to ambient air pressure, or a differential pressure sensor can be referenced against the ambient air pressure. In contrast to a system control design that uses an absolute pressure sensor, the use of gauge pressure can provide a direct link between the controlled vacuum pressure and the actual pressures and forces experienced by a user of the system. Due to its small size, differential capability, surface mount configuration, and temperature compensation, it has been determined that suitable pressure sensors include, for example, the model 26PC15SMT sensor manufactured by Honeywell International Inc. of Morris Plains, N.J., U.S.A. Other acceptable sensors are also available.

In another embodiment of semi-automatic operation, a vacuum pump can be preset to draw a particular level of vacuum once activated. Therefore, the single intermittent push of a push-button or other actuator can cause the vacuum pump to operate until an associated pressure sensor determines that the desired pressure has been met. It is also possible to mix modes of operation by allowing the user to select a semi-automatic mode or a manual mode. For example, a semi-automatic mode can be entered with a quick contact of the actuator, and a manual mode can be entered by prolonged contact with the actuator.

Referring again to FIG. 12, operation of the evacuation device 80 can be enhanced by adding either logic, analog controls, or a microprocessor 502. For example, the microprocessor 502 can be configured to monitor socket pressure and automatically maintain the socket pressure within a patient or practitioner defined range of acceptable pressures. This automatic mode of operation can eliminate the need for the user to monitor the socket pressure. Moreover, the prosthetic limb then can be donned and forgotten until removal thereof is desired. It can be appreciated that such a vacuum suspension system can automatically react to conditions within the socket 78 in a manner appropriate for the user, and in ways not possible for a mechanical pump design.

The addition of sensors and a microprocessor 502 to the evacuation device 80 permits the monitoring of various conditions or parameters of the prosthetic limb and/or the user. For example, by appropriately locating a basic pressure transducer in the prosthetic socket 78, the measuring and tracking of various pressure values associated with the prosthetic socket 78 becomes possible. Pressure values of interest may include maximum or minimum socket pressure, the average pressure in the socket over some period of time, and the Root Mean Square (RMS) pressure over a defined period of time.

The period of time monitored can depend on the conditions that the user or a practitioner is evaluating. For initial setup and function testing, for example, the time period might be set to a single step. For evaluation on more complicated tasks such as engaging in a sport or ascending/descending stairs, the time period might be extended to obtain a target range for all of the various ways that the activity at issue might be performed. The test period can be extended to a period of days to track values for the user's entire range of activities. Another parameter that may be tracked is some measure of the amount of pressure the user is exposed to over the course of a period of time. Measure of this parameter can be the integral of pressure as a function of time, or the integral of the pressure squared as a function of time. With an appropriate link to the microprocessor 502, such data can then be displayed on a PC, a key fob device 420, or some other display unit for viewing and analysis by the user and/or practitioner. The data may also be saved for later reference.

The quality of the seal of any vacuum-based prosthetic suspension system may be monitored. While it is difficult to directly monitor the seal, the duty cycle of an automatically-controlled vacuum pump motor can be monitored as the vacuum pump acts to maintain the vacuum level within the prosthetic socket. Increases in the duty cycle can indicate increases in air leaks and a degradation of the seal. To properly monitor this condition, a base line vacuum pump duty cycle can be obtained during setup of the associated prosthesis. Monitoring the duty cycle and comparing it to this baseline can provide a measure of the seal and allow the seal quality to be monitored.

Another mode of monitoring the prosthetic socket can be a high speed real time mode. In high speed real time mode, vacuum level variations within the socket can be monitored in real time, as they occur. Data can be recorded relative to a known time base, which can allow vacuum fluctuations to be ascribed to specific events during the user's activities. The high speed real time mode can allow graphical displays to be constructed that visualize the relationship between a user's activities and the vacuum level within the socket.

In microprocessor-equipped embodiments wherein vacuum level within the socket can be monitored, the range or variation of the vacuum level can be monitored. Accordingly, judgments as to the user's activity level can be made based thereon. In this manner, it is possible to automatically adjust the level of vacuum to the level of activity of the user. For example, the vacuum level may be increased over the typical level for a user who becomes very active. Similarly, vacuum level may be automatically decreased if a user is substantially sedentary or non-ambulatory for some period of time, and then may be automatically increased when the user becomes more active. This method of monitoring the level of user activity and automatically adjusting the vacuum to a correlating level can result in a system that continually attempts to keep the vacuum level in the socket at an appropriate level.

Moreover, different phases of an amputee's gait cycle subject the socket 78 of a prosthetic leg (or arm) to different stresses, strains, accelerations, and impacts. Thus, during different phases of the gait cycle, the pressure in the socket 78 and the sensations that the amputee experiences can differ. For example, a level of vibration that would be noticeable during the free swing phase of gait, where vibrations are at a minimum, may not be noticeable if it occurs at the point of heel strike where other masking sensations are present.

Also, drawing a vacuum during the free swing phase of the gait cycle can be more difficult to achieve and can requires more electrical energy than drawing a vacuum during the stance phase of the gait cycle. It is believed that this is due to the socket being in tension during the swing phase. During the stance phase, the socket can be driven back onto the amputee's residual limb, which can force air from the socket 78. For at least these reasons, it can be advantageous to monitor a lower limb amputee's gait cycle. Movement of the upper limb of an upper extremity amputee can be similarly monitored. Tracking can be achieved by observing the pressure fluctuations in the socket 78 and reacting thereto. When more reliable gait or other movement synchronization is desired, more complex evaluations can be achieved through the addition of accelerometers, gyroscopes, force sensors, or some combination thereof.

The use of a pushbutton, magnetic switch, and other simplistic actuators has been described above with respect to manually operable evacuation device embodiments of the present disclosure. However, other forms of evacuation device interfaces may also be used, whether in conjunction with such actuators or in place thereof.

In one embodiment of an information-only interface, basic power, pressure, and functional information can be communicated to the user through simple LED indicators. Such an interface may continually display information, or it may display information only when the patient requests it in order to conserve power. Such a display can be built into the evacuation device housing 82.

In another embodiment of an information-only interface, basic information regarding evacuation device function, etc., can be communicated to the user by means of an audio transducer. Accordingly, information can be communicated without requiring the user to view the evacuation device 80 or some other display unit associated therewith.

Pushbuttons (and similar switch-type devices) may be used in an operating and/or programming interface. Pushbuttons can be configured to draw no power when they are not actuated. Used with a properly designed low-power microprocessor, pushbuttons can account for very little power consumption. There are a number of types of switches or switch-type devices that can be used such as, for example, standard contact switches. Membrane-type switches may be a reliable, attractive, and space efficient alternative. Also, proximity or capacitive detection switches can be used to detect “touches” through a closed container and, as such, can eliminate the need for a passage from the outside of an evacuation device housing or prosthetic component to the inside. Another possibility can include a Hall-type device that operates by using a magnetic key, which can used to provide simple on/off control, or as a backup to other interfaces.

In some embodiments, such as the semi-automatic and automatic versions described above, an interface may comprise of a series of pushbuttons associated with the evacuation device 80. The pushbuttons may be located, for example, on the evacuation device housing 82. The interface can require the patient to remove clothing, or possibly cosmetic fittings, to activate the vacuum pump 84, update a program, or make changes to the vacuum settings.

In order to impart a more lifelike appearance thereto, amputees can finish their prosthesis with a cosmetic covering, which may be made of foam or other materials. However, as mentioned above, the application of a cosmetic covering to a prosthesis can inhibit access to certain embodiments of an evacuation system of the present disclosure, such as may be required for recharging, reprogramming, etc. Attempting to access such evacuation systems may be difficult and can result in damage to the cosmetic covering.

As such, in some embodiments, a programming/recharging cable 500 can be routed from the vacuum system controller to an unobtrusive and easier to access location, such as an ankle or inner thigh portion of the prosthesis. The free end of the programming/recharging cable 500 can be provided with an appropriate connector configured to be connected to a programming and/or recharging device.

Notwithstanding the functionality of the foregoing exemplary embodiments, another method of interfacing with the evacuation device 80 can use a wireless link. Thus, the evacuation device 80 may include a radio, cellular or some other form of wireless transmitter/receiver. A wireless link with the transmitter may then be established in any of several ways.

In one embodiment, a stand-alone communication device is used to communicate with the evacuation device 80. Such a stand-alone communication device may be embodied in a hand held controller 420, such as a fob, which may include, among other things, an integrated transmitter/receiver, input keys, and an alphanumeric and/or graphical display. Accordingly, the fob can be stored in a pocket and can communicate with the evacuation device 80 easily and inconspicuously. The hand held controller 420 can allow the user to observe actual operating conditions and parameters associated with the evacuation device 80 and/or prosthesis, and to modify evacuation device operation to suit their needs.

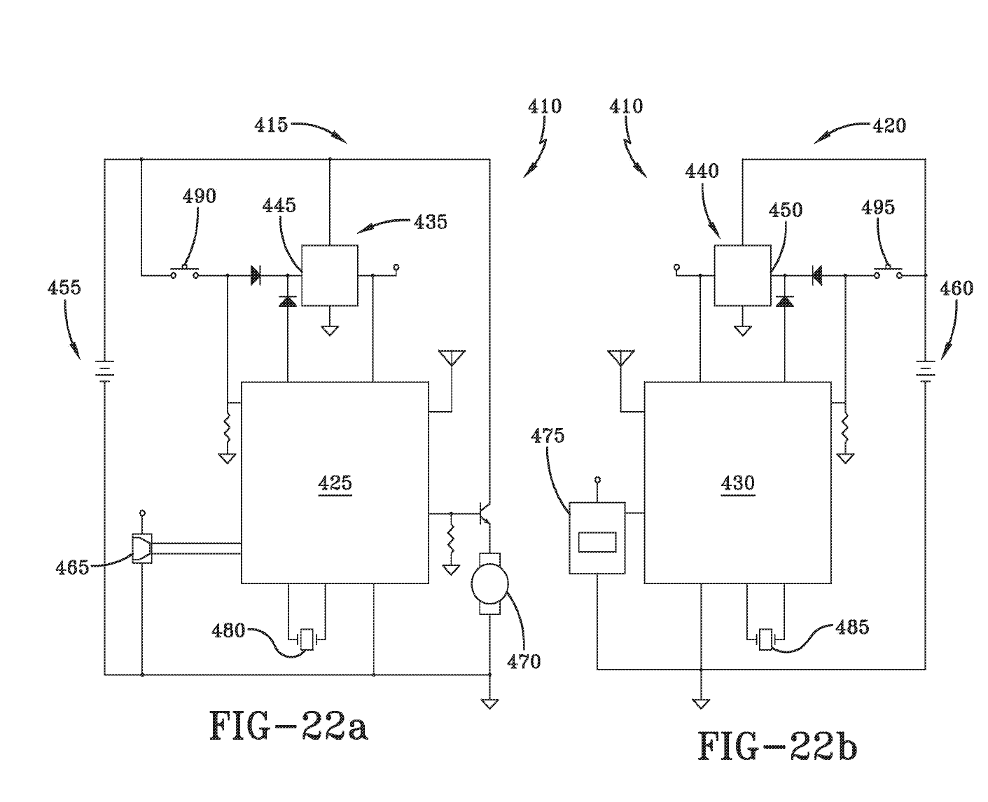

Referring collectively to FIGS. 22a and 22b, a wireless communication-based control system 410 for an evacuation device can be provided. The vacuum control assembly 415 of the control system 410 can be associated with a prosthesis. The control system 410 can include the hand held controller 420 (e.g., fob). The vacuum control assembly 415 and the hand held controller 420 can be wirelessly linked.

Wireless communication can occur via a wireless (e.g., radio) transceiver portion that is integral to a microprocessor unit 425, 430 located in the vacuum control assembly 415 and hand held controller 420, respectively. A number of microprocessors with integrated transceivers are commercially available, and would be known to one skilled in the art. In an alternative embodiment, a functional control system for an evacuation device of the present invention could be built using transceivers that are separate from their associated microprocessors. However, the integrated design can cost less, can weigh less, and can reduce circuit complexity.

Each of the vacuum control assembly 415 and hand held controller 420 can include a regulator 435, 440 with an enable pin 445, 450, a power source 455, 460, and a self-latching power supply system. Additionally, the vacuum control assembly 415 can include a pressure sensor 465 and vacuum pump 470. The hand held controller 420 can include a display 475. The display 475 of the hand held controller 420 can include a liquid crystal display (LCD), or any other display types with reduced power consumption. Suitable LCD type displays can include the model DV40311 LCD display manufactured by Densitron Displays of Santa Fe Springs, Calif., U.S.A.

Reduced power consumption can be facilitated by selection of the microprocessors 425, 430 and timing crystals 480, 485 that are respectively associated with each of the vacuum control assembly 415 and hand held controller 420. For example, a number of low lower power consuming timing crystals are available, such as, for example, the model FC-135 32.7680KA-A3 crystal manufactured by Epson Electronics America, Inc. in Wakefield, Mass., U.S.A.

The use of a regulator 435, 440 with an enable pin 445, 450, and the driving of this pin with both a pushbutton 490, 495 and an output of the respective microprocessor 425, 430, can allow the control system 410 to fully shut down when not in use—thereby consuming very little power when not needed. When needed, actuation of the associated pushbutton 490, 495 can activate (wakes up) the respective microprocessor 425, 430. Whereafter, the respective microprocessor 425, 430 can perform the required tasks and remain active as long as necessary.

Power consumption can be further reduced by powering peripheral devices such as the display 475 of the hand held controller 420 or the pressure sensor 465 of the vacuum control assembly 415 only when the associated microprocessor 425, 430 is active. Power consumption can be even further reduced by powering such peripheral devices with separate regulators that can be switched on and off by the microprocessors 425, 430 so that the regulators are operative only when necessary.

By linking the output of the pushbuttons 490, 495 respectively associated with the enable pins 445, 450 of the regulators 435, 440 to an input of the associated microprocessor 425, 430, the pushbuttons can be used to perform multiple functions. In addition to enabling a power supply and starting a microprocessor, the functions can include communicating with a microprocessor once the microprocessor is activated. For example, actuation a pushbutton 490, 495 may place the associated microprocessor in automatic mode, which thereby acts to control the vacuum level within a prosthetic socket until a patient removes the prosthesis and/or the control system otherwise determines that automatic control is no longer needed. Once such a determination is made, the control system can shut down to conserve power.

In another wireless communication enabled embodiment, a transmitter/receiver 504 may be integrated into a communication device having a computer compatible interface, such as a serial or USB interface. Accordingly, a display and computational capabilities of a computer (e.g., a PC, laptop, pen computer, PDA, smart phone etc.) can be used. More particularly, the communication device can be connected to a computer and thereafter used to wirelessly communicate with the evacuation device. In one embodiment, a practitioner can observe variations in a user's socket pressure through a step, and from step to step, and evaluate the function of the evacuation device via wireless communication. A practitioner can also adjust the evacuation device settings, and then save the settings to a hard disk or other storage medium. Additionally, wireless communication devices can be used to interact with an evacuation device in more complex ways such as, for example, in troubleshooting and programming. It is noted that all interactions of the embodiments described herein capable of being performed locally can also be performed using a wireless link.

In another embodiment, the associated control system can be removed from the prosthesis to a remote location such as, for example, to a hand held controller 420. Placing the control system remotely may allow for the installation of simple and stable firmware at the location of the vacuum pump, which can reduce the likelihood that future upgrades to the vacuum components located in/on the prosthesis will be required. For example software upgrades and reconfigurations can be made by reprogramming or replacing the handheld held controller 420—without having to access the actual prosthesis. Further, any potential time lag associated with the use of a hand held controller 420 can be overcome by running the vacuum pump at slow speed—which would have the added benefit of reducing noise.

With respect to the power consumption of the embodiments described herein, it should be noted that several very low power consumption implementations of a functional control system can be accomplished by using components from companies such as Cypress Semiconductor Corporation, headquartered in San Jose, Calif., U.S.A. Suitable components include mixed-signal array with on-chip controller devices, which can be referred to as Programmable System-on-Chip (PSoC) devices. The PSoC devices can be designed to replace multiple traditional microcontroller-based system components with a lower cost single-chip programmable component. A non-limiting example, the Cypress model CY8C20234 PSoC can be used with the embodiments described herein due to its small size and very low power consumption.

In some embodiments, the power source(s) can be discharged to an unacceptable level, e.g., when an evacuation device is not provided with self-charging capabilities, or when a user wearing a prosthetic limb having a self-charging capable evacuation device is non-ambulatory for an extended period of time. As such, some embodiments can be equipped with a means to notify the user of a low power state and/or to take action(s) directed to preserving the power remaining in the power source.

In one embodiment, an alert to a low power state can be provided by cycling the vacuum pump. That is, by repeatedly turning the vacuum pump on and off during the evacuation process, additional vibration and noise can be generated. While such cycling can increase power consumption, the additional vibration and noise can serve as a cue to the user that there is a problem. Thus, the user can be alerted to charge the system before the power storage is completely depleted. A user may be similarly alerted to a low power situation by running the vacuum pump motor at a higher speed than normal, which can increase motor and/or vacuum pump noise and alert the user to the low power situation.

Upon detection of a low power state, a reduction in power consumption can be achieved. For example, the required vacuum level can be reduced. This method may be employed directly by a user with a manually operable evacuation device, or automatically by a microprocessor controlled device. An automatic reduction in vacuum level may also serve to notify the user of a low power situation.

In some embodiments, the wireless (e.g., radio) link may be disabled once a low power state is detected. Although minimal, such a radio link can draw power from the power source when enabled. Disabling the wireless link can also force the user to manually activate the evacuation device, thus making the low power state very apparent.

Yet another method of conserving electric power can include disabling the automatic control system. Disabling the automatic control system can stop the cycling of the evacuation device, especially if the user's activity level is increasing. Moreover, the user can be forced to interact with the evacuation device in an alternate fashion that would make the low power condition apparent. Disabling the automatic control system can also allow a user to temporarily disable the evacuation device if the user's current activity level does not necessitate vacuum suspension—thereby preserving power to adjust the vacuum level should the user's activity level change.

Other means of alerting a user to a low power condition are possible. For example, a visual and/or audible alert may be employed, such as through the use of the LED or audio transducer interfaces described above. Additionally, a vibrator could also be used to communicate a low power condition to the user.

As mentioned previously, sensors, a microprocessor, and other devices may be associated with an evacuation device to form a more advanced prosthetic evacuation system. Such systems may provide for a number of operational modes that offer various advantages in function, convenience, and privacy.

In one embodiment, a multi-speed vacuum mode can be provided. At lower levels of power consumption, the vacuum pump motor can operate at a reduced level of performance, and also at a reduced noise level. Therefore, in the multi-speed vacuum mode of operation, the user may choose between one of several predetermined levels of vacuum pump performance—with lower performance levels producing less noise and higher performance levels producing more noise. Thus, for example, if the user is in a noise sensitive environment (e.g., the theater, a library, etc.) and their activity level is relatively low, the user may choose a lower performance level to reduce noise. If noise is of little or no concern, then the user might select a higher performance level.

In addition to vacuum pump performance level selection by the user, another way to take advantage of the multi-speed vacuum mode of operation can include the use of a level of activity monitor, as described above. Such a level of activity monitor can be used to detect the level of activity of the user and to subsequently adjust the performance of the vacuum pump to an appropriate level. Accordingly, power consumption can automatically be reduced when the user is substantially sedentary.

In some embodiments, an evacuation device may be used to assist with doffing (removal) of the prosthesis to which it is installed. When removal is desired, the user first typically removes a sealing sleeve, if present, and subsequently releases the vacuum in the socket by either placing a tool therein to open a passageway along the socket interior or by opening or otherwise activating an air valve. With the vacuum released, the prosthesis can then be removed from the residual limb. To assist with the removal process, an evacuation device may employ a reversible vacuum pump or a pump coupled with a directional flow control valve to pump air back into the distal end of the socket and encourage its dislodgement from the residual limb. Alternatively, an evacuation device may use two pumps, i.e., one to evacuate the socket during donning of a prosthesis and one to pressurize the socket during doffing of the prosthesis.

It is contemplated that a system capable of both evacuating and pressurizing a socket can also be used to massage a residual limb by alternatingly creating higher and lower levels of socket pressure. Limb massage can beneficially function to increase perfusion and to force excessive fluid from the residual limb.

In addition to evacuating a prosthetic socket to impart suction suspension to a prosthesis, an evacuation device of the present invention can also have therapeutic uses. Amputees are often the victims of chronic wounds that seemingly will not heal. These wounds can be the result of operations, or the result of pressure sores. One of the dilemmas faced by amputees is how to let their stump heal when its use is often necessary to their daily activities. While this dilemma is not unique to upper or lower limb amputees, it may be more problematic for lower limb amputees because they use their residual limbs for ambulation and because their residual limbs can be subjected to more forces and pressures than are those of upper limb amputees.

Research since about 1993 has indicated that sub-atmospheric pressure can be of benefit to the healing of chronic wounds. Blood flow has been found to be augmented by treatment at reduced pressures of around 125 mmHg. Healing has been shown to be further improved by cycling the reduced pressure; such as by repeatedly applying vacuum for approximately 5 minutes, removing the vacuum for 2 minutes, and repeating.

An evacuation device of the present disclosure can be used with a sealable socket to provide such a vacuum therapy regimen. The socket may be for treatment use only and may be disposable to obviate any concerns relating to the seepage of wound fluids during treatment. Such a socket may be especially useful for the treatment of new amputees. Alternatively, the socket may be part of a prosthesis. When incorporated into the stump socket of a prosthesis, the evacuation device may be programmed to enter a therapy mode when the amputee is inactive. This may be useful when the amputee has a wound(s) or other condition(s) that will benefit from vacuum therapy.

In this embodiment, the evacuation device can be programmed or otherwise set to achieve the desired vacuum level when operated. The evacuation device can be programmed to cycle on and off in order to repeatedly apply and release the vacuum, and to maintain the vacuum level for the necessary time—whatever that time is determined to be.

In conjunction with the above discussion, it is noted that one of the causes of sores on a residual limb is excessive motion of the residual limb within a prosthetic socket. An evacuated socket can help to maintain residual limb volume, thereby reducing the tendency of the residual limb to move within a prosthetic socket. However, it is difficult to know what level of vacuum is actually necessary for a given patient at a given activity level, on a specific day. To help make such a determination, a residual limb motion sensor can be integrated into a prosthetic socket, and used to adjust the vacuum level therein. If motion over some period of time is too high, more vacuum can be drawn. If the vacuum level has been maintained, but the user's activity level has declined, the vacuum level can be slowly reduced until motion is detected. The vacuum level can then be increased as necessary until the motion ends or is maintained at a level for which the current vacuum level is appropriate. Over time, a map of activity level vs. pressure (vacuum level) can be constructed and referenced to allow for quicker vacuum adjustments.

Several types of sensors can acceptably serve as the motion sensor described above. For example, the motion sensor may be comprised of one or more Hall sensors placed in the base or wall of a prosthetic socket and one or more small magnets fastened to the tip or side of a prosthetic liner worn over the residual limb. Alternatively, the motion sensor may be comprised of a mutual inductance device that measures the mutual inductance between a coil in the base of a prosthetic socket, and a small coil placed on the tip of a prosthetic liner worn over the residual limb. In another embodiment, the motion sensor may be comprised of an ultrasonic sensor that is tuned to detect a small metal plate mounted on the tip of a prosthetic liner worn over the residual limb. Placing this sensor in the prosthetic socket can directly detect a residual limb or prosthetic liner. In yet another embodiment, the motion sensor may be comprised of a force sensor placed in the prosthetic socket or liner or an instrumented lanyard attached to the liner. Intermittent contact of the residual limb with the force sensor can indicate the occurrence of residual limb motion within the socket.

In addition to collecting data on vacuum level and motion, a microprocessor can be configured to collect other data of interest, including for example, and without limitation, the amount of time that the vacuum pump(s) are active, the amount of time the control system is in manual mode vs. automatic mode, the level of battery/capacitor charge, the number and frequency of leaks detected, the amount of time that the prosthesis has been worn, temperature (inside or outside the socket), etc. This data can then be used for a variety of purposes, such as service scheduling, warranty issues, to detect operational changes that might indicate and justify modification or replacement of the prosthesis, and others. For instance: (a) an increase in vacuum level fluctuations or in motion between the limb and socket over a period of time can indicate changes in residual limb shape that may require the fabrication of a new socket; (b) an increase in activity level associated with vacuum level fluctuations, or motion between the limb and socket over a period of time when leakage did not increase can indicate changes in activity level that may justify new prosthetic components that are appropriate for the increased activity level; (c) an increase in leak detection events may indicate that sealing elements require replacement; and (d) a decrease in pump usage, vacuum level fluctuations, or motion between the limb and socket over a period of time may indicate reduced usage of the limb due to discomfort or health problems, requiring prosthesis adjustment, replacement, or other intervention. Pump usage data can also be used to determine when servicing or replacement of the pump is required.

While various embodiments have been illustrated with respect to the case of lower limb prostheses (more primarily, below knee prostheses), the present disclosure also applies to above knee lower limb prostheses and upper limb prostheses. Furthermore, additional advantages and modifications will readily appear to those skilled in the art and are considered to be within the scope of the present disclosure.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A prosthetic device, comprising:
a prosthetic socket comprising a side wall extending from a distal end of the prosthetic socket to form an interior and an exterior of the prosthetic socket, wherein the interior of the prosthetic socket is adapted to receive a residual limb;
a vacuum passage extending through the side wall of the prosthetic socket and into the interior of the prosthetic socket; and
an evacuation device comprising an electrically powered vacuum pump and a source of electric power both contained within a common housing, wherein:
the common housing is attached to the side wall on the exterior of the prosthetic socket,
the electrically powered vacuum pump is in communication with the vacuum passage, and
the electrically powered vacuum pump draws air from the interior of the prosthetic socket, while the residual limb is received within the interior of the prosthetic socket, to evacuate the prosthetic socket.

2. The prosthetic device of claim 1, wherein the evacuation device comprises an exhaust port residing in the common housing, and wherein the air from the interior of the prosthetic socket is discharged by the electrically powered vacuum pump through the exhaust port.

3. The prosthetic device of claim 1, comprising a mounting adapter built directly into the side wall of the prosthetic socket, wherein the common housing is attached to the mounting adapter.

4. The prosthetic device of claim 3, wherein the mounting adapter is laminated into the side wall of the prosthetic socket.

5. The prosthetic device of claim 3, wherein the vacuum passage extends through the mounting adapter.

6. The prosthetic device of claim 5, comprising a sealing element installed upon the mounting adapter to seal the vacuum passage.

7. The prosthetic device of claim 1, wherein the evacuation device comprises an evacuation device vacuum passage aligned with the vacuum passage that extends through the side wall of the prosthetic socket.

8. The prosthetic device of claim 7, comprising a sealing element located around the evacuation device vacuum passage.

9. The prosthetic device of claim 1, wherein the common housing is contoured.

10. The prosthetic device of claim 1, comprising a vacuum level sensor that monitors vacuum level of the interior of the prosthetic socket and a microprocessor in communication with the evacuation device.

11. The prosthetic device of claim 10, wherein the microprocessor is programmed to use input signals from the vacuum level sensor and to operate the electrically powered vacuum pump such that the interior of the prosthetic socket is automatically evacuated to a predetermined vacuum level.

12. The prosthetic device of claim 10, wherein the microprocessor is programmed to use signals from the vacuum level sensor to continually monitor vacuum level of the interior of the prosthetic socket and to automatically operate the electrically powered vacuum pump to maintain the interior of the prosthetic socket at a predetermined vacuum level.

13. The prosthetic device of claim 10, wherein the microprocessor is programmed to use signals from the vacuum level sensor to monitor vacuum level of the interior of the prosthetic socket and to operate the electrically powered vacuum pump to automatically adjust the vacuum level to correspond to changes in a user's activity level.

14. The prosthetic device of claim 1, comprising a wireless receiver-transmitter that establishes a wireless link with the evacuation device.

15. The prosthetic device of claim 1, wherein the source of electric power is rechargeable.

16. The prosthetic device of claim 1, wherein the electrically powered vacuum pump cycles to repeatedly draw and release a vacuum within the interior of the prosthetic socket, whereby vacuum therapy is provided to the residual limb.

17. A prosthetic device, comprising:
a prosthetic socket comprising a side wall extending from a distal end of the prosthetic socket to form an interior and an exterior of the prosthetic socket, wherein the interior of the prosthetic socket is adapted to receive a residual limb;
a mounting adapter built directly into the side wall of the prosthetic socket;
a vacuum passage extending into the interior of the prosthetic socket and through the side wall of the prosthetic socket and the mounting adapter; and
an evacuation device comprising an electrically powered vacuum pump and a source of electric power both contained within a common housing, wherein:
the common housing is on the exterior of the prosthetic socket and attached to the mounting adapter,
the electrically powered vacuum pump is in communication with the vacuum passage, and
the electrically powered vacuum pump draws air from the interior of the prosthetic socket, while the residual limb is received within the interior of the prosthetic socket, to evacuate the prosthetic socket.

18. The prosthetic device of claim 17, wherein the evacuation device comprises an exhaust port residing in the common housing, and wherein the air from the interior of the prosthetic socket is discharged by the electrically powered vacuum pump through the exhaust port.

19. The prosthetic device of claim 17, wherein the mounting adapter is laminated into the side wall of the prosthetic socket.

20. A prosthetic device, comprising:
a prosthetic socket comprising a side wall extending from a distal end of the prosthetic socket to form an interior and an exterior of the prosthetic socket, wherein the interior of the prosthetic socket is adapted to receive a residual limb;
a mounting adapter built directly into the side wall of the prosthetic socket;
a vacuum passage extending into the interior of the prosthetic socket and through the side wall of the prosthetic socket and the mounting adapter; and
an evacuation device comprising an electrically powered vacuum pump, a source of electric power, and an evacuation device vacuum passage each contained within a common housing, wherein:
the common housing is on the exterior of the prosthetic socket and attached to the mounting adapter;
the evacuation device vacuum passage is aligned with the vacuum passage that extends through the side wall of the prosthetic socket and the mounting adapter;
the electrically powered vacuum pump is in communication with the vacuum passage, and
the electrically powered vacuum pump draws air from the interior of the prosthetic socket, while the residual limb is received within the interior of the prosthetic socket, to evacuate the prosthetic socket.
